SEQUENCE LISTING (45 sequences)

SEQ ID NO: 1 - protein, 379 aa, Homo sapiens
MPHSSLHPSIPCPRGHGAQKAALVLLSACLVTLWGLGEPPEHTLRYLVLHLASLQLGLLLNGVCSLAEELRHIHSRYRGSYWRTVRACLGCPLRRGALLLLSIYFYYSLPNAVGPPFTWMLALLGLSQALNILLGLKGLAPAEISAVCEKGNFNVAHGLAWSYYIGYLRLILPELQARIRTYNQHYNNLLRGAVSQRLYILLPLDCGVPDNLSMADPNIRFLDKLPQQTGDHAGIKDRVYSNSIYELLENGQRAGTCVLEYATPLQTLFAMSQYSQAGFSREDRLEQAKLFCRTLEDILADAPESQNNCRLIAYQEPADDSSFSLSQEVLRHLRQEEKEEVTVGSLKTSAVPSTSTMSQEPELLISGMEKPLPLRTDFS

SEQ ID NO: 2 - protein, 378 aa, Mus musculus
MPYSNLHPAIPRPRGHRSKYVALIFLVASLMILWVAKDPPNHTLKYLALHLASHELGLLLKNLCCLAEELCHVQSRYQGSYWKAVRACLGCPIHCMAMILLSSYFYFLQNTADIYLSWMFGLLVLYKSLSMLLGLQSLTPAEVSAVCEEKKLNVAHGLAWSYYIGYLRLILPGLQARIRMFNQLHNNMLSGAGSRRLYILFPLDCGVPDNLSVVDPNIRFRDMLPQQNIDRAGIKNRVYSNSVYEILENGQPAGVCILEYATPLQTLFAMSQDAKAGFSREDRLEQAKLFCRTLEEILEDVPESRNNCRLIVYQEPTDGNSFSLSQEVLRHIRQEEKEEVTMNAPMTSVAPPPSVLSQEPRLLISGMDQPLPLRTDLI

SEQ ID NO: 3 - protein, 241 aa, Artificial Sequence
  Description of Artificial Sequence Synthetic polypeptide
LAPAEISAVCEKGNFNVAHGLAWSYYIGYLRLILPELQARIRTYNQHYNNLLRGAVSQRLYILLPLDCGVPDNLSMADPNIRFLDKLPQQTGDHAGIKDRVYSNSIYELLENGQRAGTCVLEYATPLQTLFAMSQYSQAGFSREDRLEQAKLFCRTLEDILADAPESQNNCRLIAYQEPADDSSFSLSQEVLRHLRQEEKEEVTVGSLKTSAVPSTSTMSQEPELLISGMEKPLPLRTDFS

SEQ ID NO: 4 - protein, 242 aa, Artificial Sequence
  Description of Artificial Sequence Synthetic polypeptide
GLAPAEISAVCEKGNFNVAHGLAWSYYIGYLRLILPELQARIRTYNQHYNNLLRGAVSQRLYILLPLDCGVPDNLSMADPNIRFLDKLPQQTGDHAGIKDRVYSNSIYELLENGQRAGTCVLEYATPLQTLFAMSQYSQAGFSREDRLEQAKLFCRTLEDILADAPESQNNCRLIAYQEPADDSSFSLSQEVLRHLRQEEKEEVTVGSLKTSAVPSTSTMSQEPELLISGMEKPLPLRTDFS

SEQ ID NO: 5 - protein, 232 aa, Artificial Sequence
  Description of Artificial Sequence Synthetic polypeptide
LAPAEISAVCEKGNFNVAHGLAWSYYIGYLRLILPELQARIRTYNQHYNNLLRGAVSQRLYILLPLDCGVPDNLSMADPNIRFLDKLPQQTGDHAGIKDRVYSNSIYELLENGQRAGTCVLEYATPLQTLFAMSQYSQAGFSREDRLEQAKLFCRTLEDILADAPESQNNCRLIAYQEPADDSSFSLSQEVLRHLRQEEKEEVTVGSLKTSAVPSTSTMSQEPELLISGMEK

SEQ ID NO: 6 - protein, 233 aa, Artificial Sequence
  Description of Artificial Sequence Synthetic polypeptide
GLAPAEISAVCEKGNFNVAHGLAWSYYIGYLRLILPELQARIRTYNQHYNNLLRGAVSQRLYILLPLDCGVPDNLSMADPNIRFLDKLPQQTGDHAGIKDRVYSNSIYELLENGQRAGTCVLEYATPLQTLFAMSQYSQAGFSREDRLEQAKLFCRTLEDILADAPESQNNCRLIAYQEPADDSSFSLSQEVLRHLRQEEKEEVTVGSLKTSAVPSTSTMSQEPELLISGMEK

SEQ ID NO: 7 - protein, 14 aa, Drosophila sp.
RQIKIWFQNRMKWK

SEQ ID NO: 8 - protein, 34 aa, Human alphaherpesvirus 1
DAATATRGRSAASRPTERPRAPARSASRPRRPVE

SEQ ID NO: 9 - protein, 24 aa, Artificial Sequence
  Description of Artificial Sequence Synthetic peptide
RRIRPRPPRLPRPRPRPLPFPRPG

SEQ ID NO: 10 - protein, 7 aa, Human immunodeficiency virus
RKKRQRR

SEQ ID NO: 11 - protein, 12 aa, Human immunodeficiency virus
GRKRRQRRRTPQ

SEQ ID NO: 12 - protein, 11 aa, Human immunodeficiency virus
YGRKKRRQRRR

SEQ ID NO: 13 - protein, 19 aa, Artificial Sequence
  Description of Artificial Sequence Synthetic peptide
ALWKTLLKVLKAPKKKRKV

SEQ ID NO: 14 - protein, 14 aa, Drosophila sp.
RQIKWFQNRRMKWK

SEQ ID NO: 15 - protein, 18 aa, Unknown
  Description of Unknown SynB1 sequence
RGGRLSYSRRRFSTSTGR

SEQ ID NO: 16 - protein, 10 aa, Unknown
  Description of Unknown SynB3 sequence
RRLSYSRRRF

SEQ ID NO: 17 - protein, 12 aa, Artificial Sequence
  Description of Artificial Sequence Synthetic peptide
PIRRRKKLRRLK

SEQ ID NO: 18 - protein, 12 aa, Artificial Sequence
  Description of Artificial Sequence Synthetic peptide
RRQRRTSKLMKR

SEQ ID NO: 19 - protein, 15 aa, Flock House virus
RRRRNRTRRNRRRVR

SEQ ID NO: 20 - protein, 19 aa, Brome Mosaic virus
KMTRAQRRAAARRNRWTAR

SEQ ID NO: 21 - protein, 13 aa, Human T-lymphotropic virus 2
TRRQRTRRARRNR

SEQ ID NO: 22 - protein, 13 aa, Human immunodeficiency virus
GRKKRRQRRRPPQ

SEQ ID NO: 23 - protein, 13 aa, Artificial Sequence
  Description of Artificial Sequence Synthetic peptide
GRRRRRRRRRPPQ

SEQ ID NO: 24 - protein, 17 aa, Artificial Sequence
  Description of Artificial Sequence Synthetic peptide
KLALKLALKLALALKLA

SEQ ID NO: 25 - protein, 27 aa, Homo sapiens
MGLGLHLLVLAAALQGAWSQPKKKRKV

SEQ ID NO: 26 - protein, 27 aa, Unknown
  Description of Unknown FBP sequence
GALFLGWLGAAGSTMGAWSQPKKKRKV

SEQ ID NO: 27 - protein, 27 aa, Artificial Sequence
  Description of Artificial Sequence Synthetic peptide
GALFLGFLGAAGSTMGAWSQPKKKRKV

SEQ ID NO: 28 - protein, 27 aa, Artificial Sequence
  Description of Artificial Sequence Synthetic peptide
GALFLGFLGAAGSTMGAWSQPKSKRKV

SEQ ID NO: 29 - protein, 21 aa, Artificial Sequence
  Description of Artificial Sequence Synthetic peptide
KETWWETWWTEWSQPKKKRKV

SEQ ID NO: 30 - protein, 21 aa, Artificial Sequence
  Description of Artificial Sequence Synthetic peptide
KETWFETWFTEWSQPKKKRKV

SEQ ID NO: 31 - protein, 10 aa, Human immunodeficiency virus
GRKKRRQRRR

SEQ ID NO: 32 - protein, 8 aa, Artificial Sequence
  Description of Artificial Sequence Synthetic peptide
RRRRRRLR

SEQ ID NO: 33 - protein, 12 aa, Artificial Sequence
  Description of Artificial Sequence Synthetic peptide
RRQRRTSKLMKR

SEQ ID NO: 34 - protein, 27 aa, Artificial Sequence
  Description of Artificial Sequence Synthetic peptide
GWTLNSAGYLLGKINLKALAALAKKIL

SEQ ID NO: 35 - protein, 33 aa, Artificial Sequence
  Description of Artificial Sequence Synthetic polypeptide
KALAWEAKLAKALAKALAKHLAKALAKALKCEA

SEQ ID NO: 36 - protein, 16 aa, Drosophila sp.
RQIKIWFQNRRMKWKK

SEQ ID NO: 37 - protein, 11 aa, Human immunodeficiency virus
YGRKKRRQRRR

SEQ ID NO: 38 - protein, 8 aa, Human immunodeficiency virus
RKKRRQRR

SEQ ID NO: 39 - protein, 11 aa, Artificial Sequence
  Description of Artificial Sequence Synthetic peptide
YARAAARQARA

SEQ ID NO: 40 - protein, 11 aa, Artificial Sequence
  Description of Artificial Sequence Synthetic peptide
THRLPRRRRRR

SEQ ID NO: 41 - protein, 11 aa, Artificial Sequence
  Description of Artificial Sequence Synthetic peptide
GGRRARRRRRR

SEQ ID NO: 42 - protein, 91 aa, Homo sapiens
ATEENVKRRTHNVLERQRRNELKRSFFALRDQIPELENNEKAPKVVILKKATAYILSVQAETQKLISEIDLLRKQNEQLKHKLEQLRNSCA

SEQ ID NO: 43 - protein, 8 aa, Gallus gallus
SIINFEKL

SEQ ID NO: 44 - protein, 6 aa, Artificial Sequence
  Description of Artificial Sequence Synthetic 6xHis tag
HHHHHH

SEQ ID NO: 45 - protein, 8 aa, Artificial Sequence
  Description of Artificial Sequence Synthetic peptide
DYKDDDDK

(19) United States
(12) Patent Application Publication
(10) Pub. No.: US 2021/0277077 A1
(43) Pub. Date: Sep. 9, 2021

(54) RECOMBINANT TRANSMEMBRANE DOMAIN-DEFICIENT STING AS BIOMIMETIC PROTEIN CARRIER FOR CGAMP ENHANCED CANCER IMMUNOTHERAPY

(71) Applicant: Northeastern University, Boston, MA (US)

(72) Inventor: Jiahe Li, Medford, MA (US)

(21) Appl. No.: 17/181,884

(22) Filed: Feb. 22, 2021

Related U.S. Application Data
(60) Provisional application No. 62/979,733, filed on Feb. 21, 2020.

(51) Int. Cl. C07K 14/47; C07K 16/28; A61K 31/7084; A61K 45/06

(52) U.S. Cl. CPC C07K 14/4702; C07K 16/2818; C07K 16/2827; A61K 38/00; A61K 45/06; C07K 2317/569; C07K 2319/33; A61K 31/7084

(57) ABSTRACT

Disclosed are compositions comprising a fusion protein and a STING agonist, wherein the fusion protein comprises STINGΔTM protein fused to a cell-penetrating domain or a nanobody to deliver STING agonists. Also disclosed are methods of treating cancer, which is achieved by a administering said compositions.

### RELATED APPLICATIONS

This application claims the benefit of priority to U.S. Provisional Patent Application Ser. No. 62/979,733, filed Feb. 21, 2020.

### SEQUENCE LISTING

The instant application contains a Sequence Listing, which has been submitted electronically in ASCII format and is hereby incorporated by reference in its entirety. Said ASCII copy, created on May 24, 2021, is named NEX-07201_SL.txt and is 26,047 bytes in size.

### BACKGROUND

Activation of the stimulator of interferon genes (STING) pathway through cyclic dinucleotides (CDNs) could be used as a potent vaccine adjuvant against infectious diseases as well as to increase tumor immunogenicity towards cancer immunotherapy in solid tumors. Despite the promise of CDNs, such as cGAMP, as immune adjuvants, they suffer from several limitations: (1) CDNs exhibit fast clearance from the injection site, which may induce systemic toxicity; (2) naturally derived CDNs are susceptible to enzymatic degradation, which can lower the efficacy of adjuvanticity potential; and (3) CDNs have inefficient intracellular transport properties due to limited endosomal escape or reliance on the expression of a specific transporter protein. Hence, there is an urgent need to find new strategies for delivering CDNs.

### SUMMARY

In one aspect, the present disclosure provides a composition comprising a fusion protein and a STING agonist, wherein the fusion protein comprises STINGΔTM protein fused to a cell-penetrating domain or a nanobody. Numerous embodiments are further provided that can be applied to any aspect of the present invention described herein. For example, in some embodiments, the STINGΔTM comprises an amino acid sequence with at least 75%, 80%, 85%, 90%, 95%, 96%, 97%, 98%, 99% or 100% homology to the amino acid sequence selected from SEQ ID NOs: 3-6. In some embodiments, the cell-penetrating domain or the nanobody is fused to the N-terminus of the STINGΔTM. In some embodiments, the cell-penetrating domain comprises an amino acid sequence selected from SEQ ID NOs: 7-42. In some embodiments, the nanobody is capable of binding to a cancer cell. In some embodiments, the nanobody is capable of binding to CTLA4, PD-1, PD-L1, PD-L2, A2AR, B7-H3, B7-H4, BTLA, KIR, LAG3, TIM-3 or VISTA. In some embodiments, the STING agonist is a cytosolic cyclic dinucleotide (CDN). In some embodiments, the CDN is c-di-GMP, 3′,3′cGAMP, 2′,3′cGAMP, c-di-AMP, cAIMP, cAIMP Difluor, cAIM(PS)2 Difluor (Rp,Sp), 2′2′-cGAMP, 2′3′-cGAM(PS)2 (Rp,Sp), 3′3′-cGAMP Fluorinated, c-di-AMP Fluorinated, 2′3′-c-di-AMP, 2′3′-c-di-AM(PS)2 (Rp,RP), 2′3′-c-di-AM(PS)2, c-di-GMP Fluorinated, 2′3′-c-di-GMP, or c-di-IMP. In some embodiments, the STING agonist is a non-nucleotidyl small molecule. In some embodiments, the non-nucleotidyl small molecule is 5,6-dimethylxanthenone-4-acetic acid 7 (DMXAA), flavone-8-acetic acid, 2,7-bis(2-diethylamino ethoxy)fluoren-9-one, 10-carboxymethyl-9-acridanone, 2,7,2″,2″-dispiro[indene-1″,3″-dione]-tetrahydro dithiazolo[3,2-a:3′,2′-d]pyrazine-5,10(5aH,10aH)-dione, 4-(2-chloro-6-fluorobenzyl)-N-(furan-2-ylmethyl)-3-oxo-3,4-dihydro-2H-benzo[b][1,4]thiazine-6-carboxamide, 6-Bromo-N-(naphthalen-1-yl)benzo[d][1,3]dioxole-5-carboxamide, 3-oxo-3,4-dihydro-2H-benzo[b][1,4]thiazine-6-carboxamide, 2-oxo-2,3-dihydro-1H-pyrido[2,3-b][1,4]thiazine-7-carboxamide, 2-oxo-1,2,3,4-tetrahydroquinoline-7-carboxamide, or 2-Oxo-1,2,3,4-tetrahydroquinazoline-7-carboxamides.

In another aspect, the present disclosure provides a fusion protein comprising STINGΔTM protein fused to a cell-penetrating domain or a nanobody. Numerous embodiments are further provided that can be applied to any aspect of the present invention described herein. For example, in some embodiments, the STINGΔTM comprises an amino acid sequence with at least 75%, 80%, 85%, 90%, 95%, 96%, 97%, 98%, 99% or 100% homology to the amino acid sequence selected from SEQ ID NOs: 3-6. In some embodiments, the cell-penetrating domain or the nanobody is fused to the N-terminus of the STINGΔTM. In some embodiments, the cell-penetrating domain comprises an amino acid sequence selected from SEQ ID NOs: 7-42. In some embodiments, the nanobody is capable of binding to a cancer cell. In some embodiments, the nanobody is capable of binding to CTLA4, PD-1, PD-L1, PD-L2, A2AR, B7-H3, B7-H4, BTLA, KIR, LAG3, TIM-3 or VISTA.

In another aspect, the present disclosure provides a nucleic acid molecule that hybridizes, under stringent conditions, with the complement of a nucleic acid encoding the fusion protein disclosed herein. In another aspect, the present disclosure provides a vector comprising the nucleic acid disclosed herein.

In another aspect, the present disclosure provides a method of treating cancer or an infectious disease comprising administering the composition a fusion protein and a STING agonist, wherein the fusion protein comprises STINGΔTM protein fused to a cell-penetrating domain or a nanobody. In some embodiments, the STINGΔTM comprises an amino acid sequence with at least 75%, 80%, 85%, 90%, 95%, 96%, 97%, 98%, 99% or 100% homology to the amino acid sequence selected from SEQ ID NOs: 3-6. In some embodiments, the cell-penetrating domain or the nanobody is fused to the N-terminus of the STINGΔTM. In some embodiments, the cell-penetrating domain comprises an amino acid sequence selected from SEQ ID NOs: 7-42. In some embodiments, the nanobody is capable of binding to a cancer cell. In some embodiments, the nanobody is capable of binding to CTLA4, PD-1, PD-L1, PD-L2, A2AR, B7-H3, B7-H4, BTLA, KIR, LAG3, TIM-3 or VISTA. In some embodiments, the STING agonist is a cytosolic cyclic dinucleotide (CDN). In some embodiments, the CDN is c-di-GMP, 3′,3′cGAMP, 2′,3′cGAMP, c-di-AMP, cAIMP, cAIMP Difluor, cAIM(PS)2 Difluor (Rp,Sp), 2′2′-cGAMP, 2′3′-cGAM(PS)2 (Rp,Sp), 3′3′-cGAMP Fluorinated, c-di-AMP Fluorinated, 2′3′-c-di-AMP, 2′3′-c-di-AM(PS)2 (Rp,RP), 2′3′-c-di-AM(PS)2, c-di-GMP Fluorinated, 2′3′-c-di-GMP, or c-di-IMP. In some embodiments, the STING agonist is a non-nucleotidyl small molecule. In some embodiments, the non-nucleotidyl small molecule is 5,6-dimethylxanthenone-4-acetic acid 7 (DMXAA), flavone-8-acetic acid, 2,7-bis(2-diethylamino ethoxy)fluoren-9-one, 10-carboxymethyl-9-acridanone, 2,7,2″,2″-dispiro[indene-1″,3″-dione]-tetrahydro dithiazolo[3,2-a:3′,2′-d]pyrazine-5,10(5aH,10aH)-dione, 4-(2-chloro-6-fluorobenzyl)-N-(furan-2-yl methyl)-3-oxo-3,4-dihydro-2H-benzo[b][1,4]thiazine-6-carboxamide, 6-Bromo-N-(naphthalen-1-yl)benzo[d][1,3]dioxole-5-carboxamide, 3-oxo-3,4-dihydro-2H-benzo[b][1,4]thiazine-6-carboxamide, 2-oxo-2,3-dihydro-1H-pyrido[2,3-b][1,4]thiazine-7-carboxamide, 2-oxo-1,2,3,4-tetrahydroquinoline-7-carboxamide, or 2-Oxo-1,2,3,4-tetrahydroquinazoline-7-carboxamides. In some embodiments, the method further comprising administering an immune check point inhibitor that specifically binds to an immune checkpoint protein. In some embodiments, the immune check point protein is CTLA4, PD-1, PD-L1, PD-L2, A2AR, B7-H3, B7-H4, BTLA, KIR, LAG3, TIM-3 or VISTA. In some embodiments, the method further comprising administering a chemotherapy. In some embodiments, the chemotherapy is Olaparib. In some embodiments, the cancer has impaired STING expression. In some embodiments, the cancer is hematological malignancy, acute nonlymphocytic leukemia, chronic lymphocytic leukemia, acute granulocytic leukemia, chronic granulocytic leukemia, acute promyelocytic leukemia, acute myeloid leukemia, adult T-cell leukemia, aleukemic leukemia, a leukocythemic leukemia, basophilic leukemia, blast cell leukemia, bovine leukemia, chronic myelocytic leukemia, leukemia cutis, embryonal leukemia, eosinophilic leukemia, Gross' leukemia, Rieder cell leukemia, Schilling's leukemia, stem cell leukemia, subleukemic leukemia, undifferentiated cell leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiennoid carcinoma, carcinoma epitheliale adenoides, exophytic carcinoma, carcinoma ex ulcere, carcinoma fibrosum, gelatiniform carcinoma, gelatinous carcinoma, giant cell carcinoma, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, carcinoma villosum, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, chondrosarcoma, fibrosarcoma, lymphosarcoma, melanosarcoma, myxosarcoma, osteosarcoma, endometrial sarcoma, stromal sarcoma, Ewing's sarcoma, fascial sarcoma, fibroblastic sarcoma, giant cell sarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, telangiectaltic sarcoma, Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, non-small cell lung cancer, primary brain tumors, stomach cancer, colon cancer, malignant pancreatic insulanoma, malignant carcinoid, premalignant skin lesions, testicular cancer, lymphomas, thyroid cancer, neuroblastoma, esophageal cancer, genitourinary tract cancer, malignant hypercalcemia, cervical cancer, endometrial cancer, adrenal cortical cancer, plasmacytoma, colorectal cancer, rectal cancer, Merkel Cell carcinoma, salivary gland carcinoma, melanoma, Harding-Passey melanoma, juvenile melanoma, lentigo maligna melanoma, malignant melanoma, acral-lentiginous melanoma, amelanotic melanoma, benign juvenile melanoma, Cloudman's melanoma, S91 melanoma, nodular melanoma subungal melanoma, and superficial spreading melanoma. In some embodiments, the cancer is lung cancer, melanoma, non-small cell lung cancer, ovarian cancer. In some embodiments, the infectious disease is a viral infection, or a bacterial infection. In some embodiments, the infection is associated with COVID-19 (SARS-CoV-2), SARS-CoV, MERS-CoV, Ebola virus, influenza, cytomegalovirus, variola and group A streptococcus, or sepsis.

### BRIEF DESCRIPTION OF THE DRAWINGS

FIGS. 1A-1C show schematic of using recombinant cell-penetrating (CP)-STINGΔTM as a biologically functional platform for cGAMP delivery. FIG. 1A shows that to bypass the need for synthetic vehicles, we designed and engineered a CP-STINGΔTM by replacing the transmembrane (TM) of the full-length STING with Omomyc, a cell-penetrating mini protein. FIG. 1B shows a cartoon model illustrating how CP-STINGΔTM binds cGAMP. FIG. 1C shows that by fusing with the cell-penetrating domain, the CP-STINGΔTM is capable of penetrating cells, delivering cGAMP, and engaging with downstream proteins such as TBK1 and IRF3, that result in the production of type I IFNs.

FIGS. 2A-2E show that CP-STINGΔTM effectively internalizes cancer cells. Fluorescence microscopy imaging of internalized CP-STINGΔTM in H1944 (STING_{low}) with downregulated STING expression (FIG. 2A) and A549 (STING_{absent}) without any STING expression (FIG. 2C) (scale bar=100 μm). Flow cytometry of internalized CP-STINGΔTM in H1944 (STING_{low}) with downregulated STING expression (FIG. 2B) and A549 (STING_{absent}) without any STING expression (FIG. 2D). FIG. 2E shows that a macropinocytosis inhibitor, EIPA exhibited a dose-dependent inhibition of cell-penetrating STINGΔTM in H1944. Cells were treated with “40 μg/mL CP-STINGΔTM”+1 μg/mL cGAMP” or “40 μg/mL STINGΔTM+1 μg/mL cGAMP” for 24 hours before staining with APC-anti-FLAG.

FIGS. 3A-3E show that CP-STINGΔTM markedly enhances cGAMP delivery and STING activation in vitro. FIG. 3A shows that CP-STINGΔTM plays a chaperon role in H1994 (STING_{low}) that have down-regulated STING expression. Specifically, CXCL10 was remarkably enhanced by “10 μg/mL CP-STINGΔTM+0.25 μg/mL cGAMP” or “10 μg/mL CP-STINGΔTMΔC9 (catalytically inactive mutant)+0.25 μg/mL cGAMP” compared to 100-400 fold higher concentration of free cGAMP and 40 fold higher concentration of cGAMP delivered by Lipofectamine 2000. FIG. 3B shows that CP-STINGΔTM+cGAMP forms a functional complex in A549 (STING_{absent}), which does not express endogenous STING. Only “40 μg/mL CP-STINGΔTM+1 μg/mL cGAMP” could induce CXCL10. FIG. 3C shows that after knocking out endogenous STING in H1944 by CRISPR, CXCL10 expression was only induced by “40 μg/mL CP-STINGΔTM+1 μg/mL cGAMP” but not by the catalytic inactive “40 μg/mL CP-STINGΔTMΔC9+1 μg/mL cGAMP” or free cGAMP. FIG. 3D shows that the CXCL10 production was inhibited by the TBK1 inhibitor—MRT, which indicates that the enhanced STING signaling by CP-STINGΔTM or CP-STINGΔTMΔC9 was dependent on the TBK1, a key component in the STING pathway. FIG. 3E shows that co-delivery of CP-STINGΔTM and a synthetic, non-degradable cGAMP analog, cGAMP(PS)₂(Rp/Sp), also enhances CXCL10 production in comparison to free cGAMP(PS)₂(Rp/Sp) or 10×cGAMP(PS)₂(Rp/Sp) transfected by Lipofectamine 2000, which suggests that CP-STINGΔTM promotes the cGAMP delivery instead of protecting cGAMP from enzymatic degradation. *P<0.05; **P<0.01, ***P<0.001, ****P<0.0001. Values=mean±SEM, n=4.

FIGS. 4A-4F show that CP-STINGΔTM enhances the efficacy of cGAMP as an adjuvant. FIG. 4A shows that in murine dendritic cells DC 2.4, “40 μg/mL CP-STINGΔTM+1 μg/mL cGAMP” markedly induced CXCL10 expression as evidenced by ELISA as well as upregulated surface expression of MHC-I measured by flow cytometry. Levels of OVA-specific total IgG (FIG. 4B) and the type I IFN-associated subtype IgG2c (FIG. 4C) in groups of C57BL/6 mice (n=5). FIG. 4D shows that mice were immunized with OVA alone, or OVA mixed with 1 μg/mL free cGAMP or combinations of 40 μg/mL STINGΔTM variants with or without 1 μg/mL cGAMP on days 0 and 14 via tail-based injection. On days 21, sera from different vaccination combinations were collected for OVA-specific total IgG and IgG2c quantification. On day 21, the same cohort of mice were challenged with 1 million B16-OVA (257-264aa) subcutaneously. Data of overall tumor growth (FIG. 4E), with survival rate (FIG. 4F) at the end of the study were denoted. Values are reported as mean±SEM. Statistical analysis was performed by one-way ANOVA according to the scales of *P<0.05; **P<0.01, ***P<0.001, and ****P<0.0001.

FIGS. 5A-5E show ex vivo T cell-mediated cancer cell killing after activating the STING pathway in tumor cells. FIG. 5A shows that CFSE-labeled OT1 cells were added into B16-OVA (257-264aa) cells that were pretreated with cGAMP plus indicated STINGΔTM variants for 48 hours (˜10:1 ratio of effector T cell to tumor cells). Proliferated T cells were assayed five days later. FIG. 5B shows that representative CFSE flow cytometry data from one of four independent experiments are displayed. FIG. 5C shows quantification of T cell proliferation by CFSE staining. While the pretreatment groups “40 μg/mL CP-STINGΔTM+1 μg/mL cGAMP” and “40 μg/mL CP-STINGΔTMΔC9+1 μg/mL cGAMP” promoted T cell proliferation, the variants with deficiency in cGAMP binding or cell penetration did not. FIG. 5D shows OT1-mediated cancer cell killing. B16-OVA (257-264aa) that had been pretreated with indicated STING variants plus cGAMP for 48 hours, were cocultured with OT1 cells. After five days, nonadherence T cells were removed by washing, and the viability of adherent tumor cells was assessed by the MTT assay. Experiments were repeated three times. FIG. 5E shows upregulation of SIINFEKL-restricted MHC-I on the surface of B16-OVA (257-264aa). After treating tumor cells with 1 μg/mL cGAMP and 40 μg/mL STING variants for 48 hours, only “40 μg/mL CP-STINGΔTM+1 μg/mL” cGAMP and “40 μg/mL CP-STINGΔTMΔC9+1 μg/mL cGAMP” upregulated the expression of SIINFEKL-restricted MHC-I. Graphs are expressed as mean±SEM (n=4) and statistical analysis by one-way ANOVA according to the following scale: *P<0.05; **P<0.01, ***P<0.001, and ****P<0.0001.

FIGS. 6A-6G show combining CP-STINGΔTM/cGAMP and anti-PD-1 in a syngeneic mouse melanoma model. FIG. 6A shows that groups of C57BL/6 mice were inoculated with 1 million YUMMER 1.7 melanoma cells in the flank and when tumors reached ˜150 mm³, mice were treated with intraperitoneal injection of anti-PD-1 (200 μg per mouse) and concurrently with intratumoral injection of “100 μg/mL CP-STINGΔTM+2.5 μg/mL cGAMP” (n=5), “100 μg/mL CP-STINGΔTMΔC9+2.5 μg/mL cGAMP” (n=5), “100 μg/mL CP-STINGΔTM(R237A/Y239A)+cGAMP” (n=5), “2.5 μg/mL cGAMP only” (n=5), and vehicle control (n=4). FIG. 6B shows photos for acute responses for the treatment were taken at 72 hours after treatment. FIG. 6C that shows overall tumor growth curves were measured using clipper, and tumor volume was calculated using formulations V=(L×W×W)/2, where V is tumor volume, L is tumor length, and W is tumor width. Cellular uptake of CP-STINGΔTM (n=2) was evaluated with microscopic imaging (FIG. 6D) and flow cytometry (FIG. 6E). Expression of TNF-alpha (FIG. 6F) and IFN-gamma (FIG. 6G) induced by various treatment groups (n=3) was quantified by ELISA. Statistical analysis was performed by one-way ANOVA: *P<0.05; **P<0.01.

FIGS. 7A-7B show size exclusion chromatography and SDS-PAGE. FIG. 7A shows size exclusion chromatography (SEC) of CP-STINGΔTM, CP-STINGΔTMΔC9, CP-STINGΔTM (R237A/Y239A) and STINGΔTM in PBS buffer. FIG. 7B shows SDS-PAGE of CP-STINGΔTM (Lane 2), CP-STINGΔTMΔC9 (Lane 3), CP-STINGΔTM (R237A/Y240A) (Lane 4), STINGΔTM (Lane 5), STINGΔTMΔC9 (Lane 6), and STINGΔTM(R237A/Y239A) (Lane 7) under a denaturing condition.

FIGS. 8A-8F show that CP-STINGΔTM effectively internalizes cancer cells. Fluorescence microscopy imaging of internalized CP-STINGΔTMΔC9 in H1944 (STING_{low}) (FIG. 8A), A549 (STING_{absent}) (FIG. 8C) and ovarian cell line HeLa (FIG. 8E) (scale bar=100 μm). Flow cytometry of internalized CP-STINGΔTM in SK-MEL3 (STINGₚₒₛᵢₜᵢᵥₑ) (FIG. 8B) and SK-MEL5 (STING_{absent}) (FIG. 8D). Effects of indicated small molecule inhibitors on the cellular uptake were performed in H1944 (FIG. 8F).

FIGS. 9A-9G show immunoblotting data. FIG. 9A shows immunoblotting of endogenous STING in human and mouse cell lines. CP-STINGΔTM plays a chaperon role in enhancing cGAMP delivery and subsequent CXCL-10 production in (FIG. 9B) SK-MEL-3 (STINGₚₒₛᵢₜᵢᵥₑ) and (FIG. 9C) H2122 (STING_{low}), while CP-STINGΔTM+cGAMP forms a functional complex in (FIG. 9F) SK-MEL-5 (STING_{absent}). Similarly, quantification of MHC-I upregulation in different combinations in (FIG. 9D) H1944 (STING_{low}) and (FIG. 9E) SK-MEL-3 (STINGₚₒₛᵢₜᵢᵥₑ) indicates the chaperon role of CP-STINGΔTM. (FIG. 9G) The endogenous cGAMP is not required for enhanced delivery of exogenously administered “CP-STINGΔTM+cGAMP” in H1944 cells, in which the cGAS was knocked out by CRISPR.

FIGS. 10A-10F show vaccination data. FIG. 10A shows vaccination strategy in this study. FIG. 10B shows percentage of CD8 T cells carrying the MHC-I-SIINFEKL epitope (“SIINFEKL” disclosed as SEQ ID NO: 43) from OVA₂₅₇₋₂₆₄ₐₐ via tetramer staining. FIGS. 10C and 10D show OVA-specific IgG and IgG 2c antibody levels in mouse serum in different treatment groups were measured by ELISA. FIGS. 10E and 10F show representative plots of OVA-specific IgG and IgG2c in serum from each mouse in different treatment groups.

FIGS. 11A-11D show T cell data. FIG. 11A shows T cell stimulation and FIG. 11B shows tumor cell killing effects in OT1 and B16-GFP coculture system. FIG. 11C shows that MHC-I upregulation in B16-OVA (257-264aa) is quantified by flow cytometry. FIG. 11D shows that T cell stimulation is performed in Yummer-OVA(257-264aa) and cell viability were tested.

FIGS. 12A-12C show the effect of different treatment group. FIG. 12A shows body weight measurement in different treatment group over the course of treatment. FIG. 12B shows that CXCL10 expression induced by different treatment groups (n=3) was quantified by ELISA. FIG. 12C shows immune cell profiling via antibody staining for CD4, CD8, CD11c and CD11b in YUMMER 1.7 tumors receiving different treatment regimens.

### DETAILED DESCRIPTION

In one aspect, the present disclosure provides compositions comprising a fusion protein and a STING agonist, wherein the fusion protein comprises STINGΔTM protein fused to a cell-penetrating domain or a nanobody.

In another aspect, the present disclosure provides fusion proteins comprising STINGΔTM protein fused to a cell-penetrating domain or a nanobody.

In another aspect, the present disclosure provides methods of treating cancer comprising administering the composition a fusion protein and a STING agonist, wherein the fusion protein comprises STINGΔTM protein fused to a cell-penetrating domain or a nanobody.

Activation of the stimulator of interferon genes (STING) pathway through cyclic dinucleotides (CDNs) could be used as potent vaccine adjuvants against infectious diseases as well as to increase tumor immunogenicity towards cancer immunotherapy in solid tumors. A myriad of synthetic vehicles, including liposomes, polymers, and other nanoparticle platforms, have been developed to improve the bioavailability and therapeutic efficacy of STING agonists in preclinical mouse models. However, synthetic materials may suffer from batch-to-batch variations due to complex formulations, and can elicit side effects. In contrast, protein therapeutics such as recombinant cytokines and antibodies represent a unique therapeutic modality owing to their physical and biochemical homogeneity. In the present work, the immune adaptor STING is used as a protein-based delivery system that can efficiently encapsulate CDNs in a load-to-go manner. Moreover, through genetic fusion with a protein transduction domain, the recombinant STING can spontaneously penetrate cells to markedly enhance the delivery of CDNs in a mouse vaccination model and a syngeneic mouse melanoma model. Since certain tumor cells can evade immune surveillance via loss of STING expression, the STING platform disclosed herein can serve as a functional vehicle to restore the STING signaling in a panel of lung and melanoma cell lines with impaired STING expression. Altogether, the STING-based delivery platform disclosed herein may have implications towards targeting STING-silenced tumors as well as augmenting the efficacy of STING-based vaccine adjuvants.

The cytosolic DNA sensing pathway involving cyclic GMP-AMP synthase (cGAS) and the stimulator of interferon genes (STING) represents an essential innate immune mechanism in response to foreign pathogens. Upon detection of cytosolic DNA, the intracellular nucleic acid sensor cGAS catalyzes the productions of cyclic dinucleotides (CDNs) such as 2′3′-cyclic GMP-AMP (cGAMP), which functions as a second messenger to bind the adaptor protein STING to initiate type I interferon (IFN) production and boost dendritic cell (DC) maturation and T cell infiltration. Meanwhile, the cGAS-STING signaling pathway is profound at sensing neoplastic progression by promoting type I IFN production and initiating cytotoxic T cell-mediated anti-tumor immune response. Synthetic STING agonists can be utilized to activate the innate and adaptive immune responses as a monotherapy or in combination with immune checkpoint blockade (ICB) for cancer immunotherapy.

Despite the promise of CDNs such as cGAMP as immune adjuvants, they suffer from several limitations: (1) CDNs exhibit fast clearance from the injection site, which may induce systemic toxicity, (2) naturally derived CDNs are susceptible to enzymatic degradation, which can lower the efficacy of adjuvanticity potential, and (3) CDNs have inefficient intracellular transport properties due to limited endosomal escape or reliance on the expression of a specific transporter protein. To address these challenges, two main directions are focused on: (1) generation of novel biomaterial-based delivery systems to improve the in vivo delivery of CDNs to activate innate immune cells, and (2) discovery of new STING agonist analogs via medicinal chemistry and drug screening to confer greater chemical stability and improved pharmacokinetics.

Here, we sought to develop a new delivery system that can offer structural simplicity and modularity from the perspective of delivery vehicle design, while becoming an add-on technology by incorporating newly discovered synthetic STING agonist compounds. To this end, we uncovered an unnatural function of a recombinant STING protein that lacks the hydrophobic transmembrane (TM) domain (hereinafter referred to as STINGΔTM). Notably, following delivery via commercial transfection reagents, the STINGΔTM/cGAMP complexes can activate the STING signaling pathway even in cells without endogenous STING expression. In our present work, to bypass the need for any synthetic delivery material, we sought to engineer a protein-based carrier for STING agonists by generating a cell-penetrating STINGΔTM (CP-STINGΔTM) through genetic fusion with a cell-penetrating domain, named Omomyc. As a dominant-negative form of the human MYC oncogene, Omomyc was originally identified to target KRAS-driven tumor cells in several NSCLC xenograft mouse models. Intriguingly, in a synthetic vehicle-free mode, CP-STINGΔTM markedly enhanced delivery of cGAMP in cells, which differ in the levels of endogenous STING expression or cell type. To prove its utility in vivo, we first explored CP-STINGΔTM to enhance the delivery of cGAMP as an adjuvant in a mouse model vaccinated with chicken ovalbumin. Furthermore, in a syngeneic mouse model of melanoma we explored a combination immunotherapy regimen consisting of an ICB inhibitor, anti-PD-1 and STING agonism. Collectively, our work demonstrated the potential of repurposing the immune sensing receptor as a vehicle to encapsulate and deliver immune adjuvants towards vaccine and cancer immunotherapy development.

A protein carrier (CP-STINGΔTM) was developed for efficient cytosolic delivery of STING agonists by merging the inherent capacity of the transmembrane deleted STING (STINGΔTM) in binding cGAMP and activating the downstream STING signaling with the cell-penetrating miniprotein Omomyc. Importantly, while the N terminus of Omomyc is responsible for cell targeting, the C terminus of STINGΔTM is involved in intracellular STING functions. Additionally, the two protein domains exist as a dimer on its own. Therefore, the fusion protein consisting of CP and STINGΔTM can in theory function properly with the natural configuration and stoichiometry. To confirm the functionality and versatility of the fusion protein CP-STINGΔTM, we tested a panel of NSCLC and melanoma cancer cell lines since these two cancer types can benefit from existing immunotherapy owing to high tumor mutational burden. Intriguingly, we found that CP-STINGΔTM plays distinct roles in these cell lines depending on the levels of endogenous STING expression. Specifically, co-delivery of CP-STINGΔTM and cGAMP restores the STING signaling in cancer cells either naturally deficient for STING expression or genetically knocked out by CRISPR, indicating that CP-STINGΔTM and cGAMP forms a functional complex in this setting. To the contrary, CP-STINGΔTM serves as a chaperon to markedly promote the delivery of cGAMP in cells with down-regulated STING expression, requiring 100-fold lower concentration of cGAMP than free cGAMP in STING activation and subsequent type I IFN induction. To explore potential translation of the platform, we further confirmed potent T cell proliferation and anti-tumor immune responses ex vivo and extended the observation in vivo using a mouse model of vaccination. Finally, we investigated the translational potential of our platform in combination with the immune checkpoint blockade using a syngeneic mouse melanoma model. Collectively, our CP-STINGΔTM system may provide a new paradigm of delivering STING agonists towards vaccines and cancer immunotherapy.

In comparison to many existing synthetic delivery systems, our CP-STING protein as a delivery vehicle is unique in several aspects: (1) Instead of electrostatic complexation, which is particularly challenging to dinucleotides owing to low charge densities, we have made use of the inherent strong affinity between the C-terminus of STING and its agonist to efficiently encapsulate STING agonists. (2) The CP-STINGΔTM itself is in essence a single long polymer with a fixed degree of “polymerization”, and therefore is structurally well defined as evidenced by size exclusion chromatography and SDS-PAGE. This feature may minimize batch-to-batch variations, commonly occurring in synthetic delivery vehicles. (3) The fusion protein can be produced and purified from the standard E. coli based recombinant protein expression system in a high yield in conjunction with the low-cost metal affinity purification, which are easily accessible to many laboratories. (4) The most important feature is that CP-STINGΔTM in complex with cGAMP can form a functional complex to activate the endogenous STING signaling in cancer cells deficient for the STING expression. This attribute may have important clinical implications given that certain cancers frequently silence the expression of endogenous STING (referred to as tumor-intrinsic STING) as a mechanism to evade anti-tumor immune responses. Specifically, the loss of tumor-intrinsic STING expression has been shown to impair tumor cell antigenicity and susceptibility to lysis by tumor infiltrating lymphocytes through the downregulation of MHC class I expression on the surface of cancer cells In addition to NSCLC and melanoma, decreased expression of STING in tumor cells has been correlated with poor prognosis in patients with gastric and colon cancers. Conversely, activation of tumor-intrinsic STING signaling has been found to dictate chemotherapy-induced antitumor cytotoxic T cell responses (e.g., olaparib) in triple-negative breast cancer.

CP-STINGΔTM in the setting of systemic delivery can be characterized to optimize the dose and frequency of the fusion protein. Additionally, by employing transgenic mouse models with STING deficiency in different cell types (e.g. tumor cells versus different immune cell subtypes), we can further elucidate exact targets of CP-STINGΔTM, and therefore assess the contribution of tumor-intrinsic STING in developing anti-tumor immune responses. Finally, given the modularity of the fusion protein, we can potentially substitute the cell-penetrating domain with a more specific protein domain such as nanobody to target particular cell type or tumor microenvironment such that our fusion platform can be extended to targeted delivery of STING agonists in a manner similar to antibody drug conjugates. Alternatively, direction fusion of a nanobody such as anti-PD (L)1 with STINGΔTM may simultaneously leverage ICB and STING in a single protein format. Therefore, our approach may offer a unique direction towards the STING-based therapeutics.

### Definition

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well-known and commonly used in the art.

The terms “a,” “an” and “the” include plural referents unless the context in which the term is used clearly dictates otherwise. The terms “a” (or “an”), as well as the terms “one or more,” and “at least one” can be used interchangeably herein. Furthermore, “and/or” where used herein is to be taken as specific disclosure of each of the two or more specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

The term “comprise” is generally used in the sense of include, that is to say permitting the presence of one or more features or components. Wherever embodiments, are described herein with the language “comprising,” otherwise analogous embodiments described in terms of “consisting of,” and/or “consisting essentially of” are also provided.

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

The term “a small molecule” is a compound having a molecular weight of less than 2000 Daltons, preferably less than 1000 Daltons. Typically, a small molecule therapeutic is an organic compound that may help regulate a biological process.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

The terms “cancer,” “tumor,” “cancerous,” and “malignant” refer to or describe the physiological condition in mammals that is typically characterized by unregulated cell growth.

Examples of cancers include but are not limited to, carcinoma including adenocarcinomas, lymphomas, blastomas, melanomas, sarcomas, and leukemias. More particular examples of such cancers include squamous cell cancer, small-cell lung cancer, non-small cell lung cancer, gastrointestinal cancer, Hodgkin's and non-Hodgkin's lymphoma, pancreatic cancer, glioblastoma, glioma, cervical cancer, ovarian cancer, liver cancer such as hepatic carcinoma and hepatoma, bladder cancer, breast cancer (including hormonally mediated breast cancer, see, e.g., Innes et al., Br. J. Cancer 94:1057-1065 (2006)), colon cancer, colorectal cancer, endometrial carcinoma, myeloma (such as multiple myeloma), salivary gland carcinoma, kidney cancer such as renal cell carcinoma and Wilms' tumors, basal cell carcinoma, melanoma, prostate cancer, vulval cancer, thyroid cancer, testicular cancer, esophageal cancer, various types of head and neck cancer and cancers of mucinous origins, such as mucinous ovarian cancer, cholangiocarcinoma (liver) and renal papillary carcinoma. In particular embodiments, the cancer is breast, endometrial, or uterine cancer. In other embodiments, the cancer is a myeloma (e.g., multiple myeloma, plasmacytoma, localized myeloma, and extramedullary myeloma), or endometrial, gastric, liver, colon, renal or pancreatic cancer.

A “recombinant” polypeptide, protein or antibody refers to polypeptide, protein or antibody produced via recombinant DNA technology. Recombinantly produced polypeptides, proteins and antibodies expressed in host cells are considered isolated for the purpose of the present disclosure, as are native or recombinant polypeptides which have been separated, fractionated, or partially or substantially purified by any suitable technique.

The term “percent sequence identity” or “percent identity” between two polynucleotide or polypeptide sequences refers to the number of identical matched positions shared by the sequences over a comparison window, taking into account additions or deletions (i.e., gaps) that must be introduced for optimal alignment of the two sequences. A matched position is any position where an identical nucleotide or amino acid is presented in both the target and reference sequence. Gaps presented in the target sequence are not counted since gaps are not nucleotides or amino acids. Likewise, gaps presented in the reference sequence are not counted since target sequence nucleotides or amino acids are counted, not nucleotides or amino acids from the reference sequence. The percentage of sequence identity is calculated by determining the number of positions at which the identical amino-acid residue or nucleic acid base occurs in both sequences to yield the number of matched positions, dividing the number of matched positions by the total number of positions in the window of comparison and multiplying the result by 100 to yield the percentage of sequence identity. The comparison of sequences and determination of percent sequence identity between two sequences can be accomplished using readily available software programs. Suitable software programs are available from various sources, and for alignment of both protein and nucleotide sequences. One suitable program to determine percent sequence identity is bl2seq, part of the BLAST suite of program available from the U.S. government's National Center for Biotechnology Information BLAST web site (blast.ncbi.nlm.nih.gov). B12seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at ebi.ac.uk/Tools/psa.

STING Protein

The term “STING”, also known as stimulator of interferon genes (STING), transmembrane protein 173 (TMEM173) and MPYS/MITA/ERIS. STING is a protein that in humans is encoded by the STING1 gene. STING plays an important role in innate immunity. STING induces type I interferon production when cells are infected with intracellular pathogens, such as viruses, mycobacteria and intracellular parasites. Type I interferon, mediated by STING, protects infected cells and nearby cells from local infection by binding to the same cell that secretes it (autocrine signaling) and nearby cells (paracrine signaling.)

Below are non-limiting examples of STING proteins.

| SEQ ID NO: 1 Human STING protein | |
|---|---|
| (SEQ ID NO: 1) | |
| 1 | mhpsslhpsi pcprghgaqk aalvllsacl vtlwglgepp ehtlrylvlh laslqlglll |
| 61 | mgvcslaeel rhihsryrgs ywrtvraclg cplrrgalll lsiyfyyslp navgppftwm |
| 121 | lallglsqal nillglkgla paeisavcek gnfnvahgla wsyyigylrl ilpelqarir |
| 181 | tynqhynnll rgavsqrlyi llpldcgvpd nlsmadpnir fldklpqqtg dhagikdrvy |
| 241 | snsiyellen gqragtcvle yatplqtlfa msqysqagfs redrleqakl fcrtledila |
| 301 | dapesqnncr liayqepadd ssfslsqevl rhlrqeekee vtvgslktsa vpststmsqe |
| 361 | pellisgmek plplrtdfs |
| SEQ ID NO: 2 Mouse STING protein | |
| (SEQ ID NO: 2) | |
| 1 | mpysnlhpai prprghrsky valiflvasl milwvakdpp nhtlkylalh lashelglll |
| 61 | knlcclaeel chvqsryqgs ywkavraclg cpihcmamil lssyfyflqn tadiylswmf |
| 121 | gllvlyksls mLlglqsltp aevsavceek klnvahglaw syyigylrli lpglqarirm |
| 181 | fnqlhnnmls gagsrrlyil fpldcgvpdn lsvvdpnirf rdmlpqqnid ragiknrvys |
| 241 | nsvyeileng qpagvciley atplqtlfam sqdakagfsr edrleqaklf crtleeiled |
| 301 | vpesrnncrl ivyqeptdgn sfslsqevlr hirqeekeev tmnapmtsva pppsvlsqep |
| 361 | rllisgmdqp lplrdtli |
| SEQ ID NO: 3 Human STINGΔTM protein | |
| (SEQ ID NO: 3) | |
| 1 | lapaeisavc ekgnfnvahg lawsyyigyl rlilpelqar irtynqhynn llrgavsqrl |
| 61 | yillpldcgv pdnlsmadpn irfldklpqq rgdhagikdr vysnsiyell engqragtcv |
| 121 | leyatplqtl famsqysqag gsredrleqa klfcrtledi ladapesqnn crliayqepa |
| 181 | ddssfslsqe vlrhlrqeek eevtvgslkt savpststms qepellisgm ekplplrtdf |
| 241 | s |
| SEQ ID NO: 4 Mouse STINGΔTM protein | |
| (SEQ ID NO: 4) | |
| 1 | glapaeisav cekgnfnvah glawsyyigy lrlilpelqa rirtynqhyn nllrgavsqr |
| 61 | lyillpldcg vpdnslmadp nirfldklpq qtgdhagikd rvysnsiyel lengqragtc |
| 121 | vleyatplqt lfamsqysqa gfsredrleq aklfcrtled iladapesqn ncrliayqep |
| 181 | addssfslsq evlhrlrqee keevtvgslk tsavpststm sqepellisg mekplplrtd |
| 241 | fs |
| SEQ ID NO: 5 Human STINGΔTMΔC9 | |
| (SEQ ID NO: 5) | |
| 1 | lapaeisavc ekgnfnvahg lawsyyigyl rlilpelqar irtynqhynn llrgavsqrl |
| 61 | yillpldcgv pdnlsmadpn irfldklpqq tgdhagikdr vysnsiyell engqragtcv |
| 121 | leyatpdqtl famsqysqag fsredrleqa klfcrtledi ladapesqnn crliayqppa |
| 181 | ddssfslsqe vlrhlrqeek eevtvgslkt savpststms qepellisgm ek |
| SEQ ID NO: 6 Mouse STINGΔTMΔC9 | |
| (SEQ ID NO: 6) | |
| 1 | glapaeisav cekgnfnvah glawsyyigy lrlilpelqa rirtynqhyn nllrgavsqr |
| 61 | lyillpldcg vpdnlsmadp nirfldklpq qtgdhagikd rvysnsiyel lengpragtc |
| 121 | vleyatplqt lfamsqysqa gfsredrleq aklfcrtled iladapesqn ncrliayqep |
| 181 | addssfslsq evlrhlrqee keevtvgslk tsavpststm sqepellisg mek |

Cell-Penetrating Peptides

The term “cell-penetrating peptide sequence” is used in the present specification interchangeably with “CPP”, “protein transducing domain” or “PTD”. It refers to a peptide chain of variable length that directs the transport of a protein inside a cell. The delivering process into cell commonly occurs by endocytosis but the peptide can also be internalized into cell by means of direct membrane translocation. CPPs typically have an amino acid composition that either contains a high relative abundance of positively charged amino acids such as lysine or arginine or has sequences that contain an alternating pattern of polar/charged amino acid and non-polar, hydrophobic amino acids.

Cell-penetrating peptides (CPPs) are short peptides that facilitate cellular intake and uptake of molecules ranging from nanosize particles to small chemical compounds to large fragments of DNA. The “cargo” is associated with the peptides either through chemical linkage via covalent bonds or through non-covalent interactions. CPPs deliver the cargo into cells, commonly through endocytosis.

CPPs typically have an amino acid composition that either contains a high relative abundance of positively charged amino acids such as lysine or arginine or has sequences that contain an alternating pattern of polar, charged amino acids and non-polar, hydrophobic amino acids. These two types of structures are referred to as polycationic or amphipathic, respectively. A third class of CPPs are the hydrophobic peptides, containing only apolar residues with low net charge or hydrophobic amino acid groups that are crucial for cellular uptake.

Numerous CPPs are known in the art, any of which can be part of the heterologous fusion proteins of the present invention. Some examples of CPPs known in the art are provided herein.

Examples of CPPs that can be used in the present invention include, without limitation:

the CPP found in Drosophila antennapedia protein (RQIKIWFQNR MKWK. SEQ ID NO: 7),
the CPP found in the herpesvirus simplex 1 (HSV-11) VP22 DNA-binding protein
(DAATATRGRSAASRPTERPRAPARSASRPRRPVE, SEQ ID NO: 8),
the CPP of Bac-7 (RRIRPRPPRLPRPRPRPLPFPRPG; SEQ ID NO: 9),
the CPPs of the HIV-1 TAT protein consisting of amino acids 49-57 (RKK RQRR,
SEQ ID NO: 10),
amino acids 48-60 (GRK RRQRRRTPQ, SEQ ID NO: 11),
amino acids 47-57 (YGRKKRRQRRR; SEQ ID NO: 12),
the CPP of S413-PV peptide (ALWKTLLK VLKAPKKKRKV; SEQ ID NO: 13),
the CPP of penetratin (RQIKWFQNRRMKWK; SEQ ID NO: 14),
the CPP of SynB1 (RGGRLSYSRRRFSTSTGR; SEQ ID NO: 15),
the CPP of SynB3 (RRLSYSRRRF; SEQ ID NO: 16),
the CPP of PTD-4 (PIRRRKKLRRLK; SEQ ID NO: 17),
the CPP of PTD-5 (RRQRRTSKLMKR; SEQ ID NO: 18),
the CPP of the FHV Coat-(35-49) (RRRRNRTRRNRRRVR; SEQ ID NO: 19),
the CPP of BMV Gag-(7-25) (KMTRAQRRAAARRNRWTAR; SEQ ID NO: 20),
the CPP of HTLV-II Rex-(4-16) (TRRQRTRRARRNR; SEQ ID NO: 21),
the CPP of D-Tat (GRKKRRQRRRPPQ, SEQ ID NO: 22),
the CPP R9-Tat (GRRRRRRRRRPPQ; SEQ ID NO: 23),
the CPP of MAP (KLALKLALKLALALKLA; SEQ ID Na 24),
the CPP of SBP (MGLGLHLLVLAAALQGAWSQPKKKRKV; SEQ ID NO: 25),
the CPP of FBP (GALFLGWLGAAGSTMGAWSQPKKKRKV; SEQ ID NO: 26),
the CPP of MPG (ac-GALFLGFLGAAGSTMGAWSQPKKKRKV-cya; SEQ ID NO: 27),
the CPP of MPG(ENLS) (ac-GALFLGFLGAAGSTMGAWSQPKSKRKV-cya; SEQ ID NO: 28),
the CPP of Pep-1 (ac-KETWWETWWTEWSQPKKKRKV-cya; SEQ ID NO: 29),
the CPP of Pep-2 (ac-KETWEETWFTEWSQPKKKRKV-cya; SEQ ID NO: 30),
the GRKKRRQRRR sequence (SEQ ID NO: 31),
the RRRRRRLR sequence (SEQ ID NO: 32),
the RRQRRTS MAWR sequence (SEQ ID NO: 33),
Transportan GWTLNSAGYLLGKINLKALAALAKKIL (SEQ ID NO: 34),
KALAWEAKLAKALAKALAKHLAKALAKALKCEA (SEQ ID NO: 35),
RQIKIWFQNRRMKWKK (SEQ ID NO: 36),
the YGRKKRRQRRR sequence (SEQ ID NO: 37),
the RKKRRQRR sequence (SEQ ID NO: 38),
the YARAAARQARA sequence (SEQ ID NO: 39),
the THRLPRRRRRR sequence (SEQ ID NO: 40),
the GORRARRRRRR sequence (SEQ ID NO: 41),
the Omomyc CPP (SEQ ID NO:42),
1 ATEENVKRRT HNVLERQRRN ELKRSFFALR DQIPELENNE KAPKVVILKK ATAYILSVQA
61 ETQKLISEID LLRKQNEQLK HKLEQLRNSC A (SEQ ID NO: 42)

Nanobody

A single-domain antibody (sdAb), also known as a nanobody, is an antibody fragment consisting of a single monomeric variable antibody domain. Like a whole antibody, it is able to bind selectively to a specific antigen. With a molecular weight of only 12-15 kDa, single-domain antibodies are much smaller than common antibodies (150-160 kDa) which are composed of two heavy protein chains and two light chains, and even smaller than Fab fragments (˜50 kDa, one light chain and half a heavy chain) and single-chain variable fragments (˜25 kDa, two variable domains, one from a light and one from a heavy chain).

Given the modularity of the fusion protein of the present invention, a more specific protein domain such as nanobody can be fused to STINGΔTM to target particular cell type or tumor microenvironment such that our fusion platform can be extended to targeted delivery of STING agonists in a manner similar to antibody drug conjugates. Alternatively, direction fusion of a nanobody such as anti-PD-L1 with STINGΔTM may simultaneously leverage ICB and STING in a single protein format. Examples of nanobodies include, but are not limited to, anti-CTLA4 antibody, anti-PD-1 antibody, anti-PD-L1 antibody, anti-PD-L2 antibody, anti-A2AR antibody, anti-B7-H3 antibody, anti-B7-H4 antibody, anti-BTLA antibody, anti-KIR antibody, anti-LAG3 antibody, anti-TIM-3 antibody or anti-VISTA antibody.

STING Agonist

STING (also known as TMEM173, MITA, ERIS, and MPYS) is an endoplasmic reticulum (ER) dimeric adaptor protein with 42 kDa 379 amino acids (aa). It contains a transmembrane region (TM1-4, aa 1-154), a cyclic dinucleotide (CDN)-binding domain (CBD, aa 155-341) and a C-terminal tail (CTT, aa 342-379).

Many types of cancers can induce a spontaneous adaptive T cell response, and foster an immunosuppressive microenvironment favoring its development. Therefore, targeting the cGAS-STING-TBK1 pathway by using agonists to “heat up” tumor microenvironment via secretion of IFNs and other cytokines would enhance anti-tumor immune response. Recent years have witnessed the rapid advances in the development of CDN analogues or non-nucleotidyl small molecules as STING agonists to mimetic functions of the endogenous 2′,3′-cGAMP.

U.S. Pat. Nos. 10,604,542, 10,723,756, 10,703,738, 10,759,825, 10,562,929, 10,730,907, and 10,793,557, US applications US2021/0008190, US2020/0330427, and US2020/0113924, and PCT application WO2019/183578 describe STING agonists. Each of these publications is hereby incorporated by reference in its entirety, and in particular for the STING agonists described therein.

Examples of STING agonists include, but are not limited to:

(1) Natural and synthetic CDNs as direct STING agonists: c-di-GMP, 3′,3′cGAMP, 2′,3′cGAMP, c-di-AMP, cAIMP, cAIMP Difluor, cAIM(PS)2 Difluor (Rp,Sp), 2′2′-cGAMP, 2′3′-cGAM(PS)2 (Rp,Sp), 3′3′-cGAMP Fluorinated, c-di-AMP Fluorinated, 2′3′-c-di-AMP, 2′3′-c-di-AM(PS)2 (Rp,RP), 2′3′-c-di-AM(PS)2, c-di-GMP Fluorinated, 2′3′-c-di-GMP, or c-di-IMP.

(2) Non-nucleotidyl small molecule STING agonists: 5,6-dimethylxanthenone-4-acetic acid 7 (DMXAA), flavone-8-acetic acid, 2,7-bis(2-diethylamino ethoxy)fluoren-9-one, 10-carboxymethyl-9-acridanone, 2,7,2″,2″-dispiro[indene-1″,3″-dione]-tetrahydro dithiazolo[3,2-a:3′,2′-d]pyrazine-5,10(5aH,10aH)-dione, 4-(2-chloro-6-fluorobenzyl)-N-(furan-2-yl methyl)-3-oxo-3,4-dihydro-2H-benzo[b][1,4]thiazine-6-carboxamide, 6-Bromo-N-(naphthalen-1-yl)benzo[d][1,3]dioxole-5-carboxamide, 3-oxo-3,4-dihydro-2H-benzo[b][1,4]thiazine-6-carboxamide, 2-oxo-2,3-dihydro-1H-pyrido[2,3-b][1,4]thiazine-7-carboxamide, 2-oxo-1,2,3,4-tetrahydroquinoline-7-carboxamide, or 2-Oxo-1,2,3,4-tetrahydroquinazoline-7-carboxamides.

Preparation of Fusion Proteins Comprising STINGΔTM Protein Fused to a Cell-Penetrating Domain or a Nanobody

The fusion proteins comprising STINGΔTM protein fused to a cell-penetrating domain or a nanobody of the compositions may be produced by either synthetic chemical processes or by recombinant methods or a combination of both methods. The fusion proteins comprising STINGΔTM protein fused to a cell-penetrating domain or a nanobody may be prepared as full-length polymers or be synthesized as non-full length fragments and joined. Chemical synthesis of peptides is routinely performed by methods well known to those skilled in the art for either solid phase or solution phase peptide synthesis. For solid phase peptide synthesis, so called t-Boc (tert-Butyloxy carbonyl) and Fmoc (Fluorenyl-methoxy-carbonyl) chemistry, referring to the N-terminal protecting groups, on polyamide or polystyrene resin have become the conventional methods (Merrifield, R B. 1963 and Sheppard, R C. 1971, respectively). Unlike ribosomal protein synthesis, solid-phase peptide synthesis proceeds in a C-terminal to N-terminal fashion. The N-termini of amino acid monomers is protected by these two groups and added onto a deprotected amino acid chain. Deprotection requires strong acid such as trifluoroacetic acid for t-Boc and bases such as piperidine for Fmoc. Stepwise elongation, in which the amino acids are connected step-by-step in turn, is ideal for small peptides containing between 2 and 100 amino acid residues.

Non-naturally occurring residues may be incorporated into sion proteins comprising STINGΔTM protein fused to a cell-penetrating domain or a nanobody. Examples of non-ribosomally installed amino acids that may be used in accordance with a present invention and still form a peptide backbone include, but are not limited to: D-amino acids, β-amino acids, pseudo-glutamate, γ-aminobutyrate, ornithine, homocysteine, N-substituted amino acids (R. Simon et al., Proc. Natl. Acad. Sci. U.S.A. (1992) 89: 9367-71; WO 91/19735 (Bartlett et al.; incorporated by reference), U.S. Pat. No. 5,646,285 (Baindur; incorporated by reference), α-aminomethyleneoxy acetic acids (an amino acid-Gly dipeptide isostere), and α-aminooxy acids and other amino acid derivatives having non-genetically non-encoded side chain function groups etc. Peptide analogs containing thioamide, vinylogous amide, hydrazino, methyleneoxy, thiomethylene, phosphonamides, oxyamide, hydroxyethylene, reduced amide and substituted reduced amide isosteres and β-sulfonamide(s) may be employed.

In another process, unnatural amino acids have been introduced into recombinantly produced proteins by a method of codon suppression. In one aspect, the use of codon suppression techniques could be adapted to introduce an aldehyde or ketone functional group or any other functional group in any suitable position within a polypeptide chain for conjugation (see e.g. WO 2006/132969; incorporated by reference).

Alternatively, recombinant expression methods are particularly useful. Recombinant protein expression using a host cell (a cell artificially engineered to comprise nucleic acids encoding the sequence of the peptide and which will transcribe and translate, and, optionally, secrete the peptide into the cell growth medium) is used routinely in the art. For recombinant production process, a nucleic acid coding for the amino acid sequence of the peptide would typically be synthesized by conventional methods and integrated into an expression vector. Such methods are particularly preferred for manufacture of the polypeptide compositions comprising the peptides fused to additional polypeptide sequences or other proteins or protein fragments or domains. The host cell can optionally be at least one selected from E. coli, COS-1, COS-7, HEK293, BHK21, CHO, BSC-1, Hep G2, 653, SP2/0, 293, HeLa, myeloma, lymphoma, yeast, insect or plant cells, or any derivative, immortalized or transformed cell thereof. Also provided is a method for producing at least one peptide, comprising translating the peptide encoding nucleic acid under conditions in vitro, in vivo or in situ, such that the peptide is expressed in detectable or recoverable amounts. The techniques well known in the art, see, e.g., Ausubel, et al., ed., Current Protocols in Molecular Biology, John Wiley & Sons, Inc., NY, N.Y. (1987-2001); Sambrook, et al., Molecular Cloning: A Laboratory Manual, 2nd Edition, Cold Spring Harbor, N.Y. (1989).

Methods of fusing antibodies like nanobodies with proteins is known in the art, see, e.g., LaFleur, et al., MAbs. 2013 March-April; 5(2):208-18. Small binding domains can be fused to multiple locations on antibodies and still retain binding affinity to ligand and antigen.

Nucleic Acids Encoding Fusion Protein Comprising STINGΔTM Protein Fused to a Cell-Penetrating Domain or a Nanobody and their Expression

Nucleic acid molecules and combinations of nucleic acid molecules that encode a fusion protein comprising STINGΔTM protein fused to a cell-penetrating domain or a nanobody are also provided. In some embodiments, the nucleic acids molecules encode a fusion protein comprising STINGΔTM protein fused to a cell-penetrating domain or a nanobody.

The nucleic acid molecules disclosed herein can be in the form of RNA or in the form of DNA. DNA includes cDNA, genomic DNA, and synthetic DNA; and can be double-stranded or single-stranded, and if single stranded can be the coding strand/or non-coding (anti-sense) strand.

In certain embodiments, the nucleic acid molecule is isolated. In additional embodiments, a nucleic acid molecule is substantially pure. In some embodiments, the nucleic acid is cDNA or is derived from cDNA. In some embodiments, the nucleic acid is be recombinantly produced.

In some embodiments, the nucleic acid molecule comprises a fusion protein comprising STINGΔTM protein fused to a cell-penetrating domain or a nanobody coding sequence operably linked to a control sequence that controls the expression of the coding sequence in a host cell or in vitro. In particular embodiments, the coding sequence is a cDNA. The disclosure also relates to vectors containing nucleic acid molecules comprises a fusion protein comprising STINGΔTM protein fused to a cell-penetrating domain or a nanobody coding sequence operably linked to a control sequence that controls the expression of the coding sequence in a host cell or in vitro.

A host cell may be a cell or a population of cells harboring or capable of harboring a recombinant nucleic acid. Host cells can be prokaryotic (e.g., E. coli), or eukaryotic. The host cells can be fungal cells including yeast such as Saccharomyces cerevisiae, Pichia pastoris, or Schizosaccharomyces pombe. The host cells also be any of various animal cells, such as insect cells (e.g., Sf-9) or mammalian cells (e.g., HEK293F, CHO, COS-7, NIH-3T3, NS0, PER.C6®, and hybridoma). In further embodiments, the host cells is a CHO cell selected from CHO-K, CHO-0, CHO-Lec10, CHO-Lec13, CHO-Lec1, CHO Pro⁻⁵, and CHO dhfr⁻. In particular embodiments, the host cell is a hybridoma.

In some embodiments, the disclosure provides isolated nucleic acids such as a fusion protein comprising STINGΔTM protein fused to a cell-penetrating domain or a nanobody encoding cDNA fragments, sufficient for use as a hybridization probe, PCR primer or sequencing primer.

A vector may be a construct, which is capable of delivering, and in some embodiments, expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

Pharmaceutical Compositions and Administration Methods

Methods of preparing and administering a composition comprising a fusion protein and a STING agonist, wherein the fusion protein comprises STINGΔTM protein fused to a cell-penetrating domain or a nanobody. The methods of administering the composition to a subject in need thereof are known to or are readily determined by those of ordinary skill in the art. The route of administration of the composition can be, for example, oral, parenteral, by inhalation or topical. The term parenteral includes, e.g., intravenous, intraarterial, intraperitoneal, intramuscular, intraocular, subcutaneous, rectal, or vaginal administration. While all these forms of administration are clearly contemplated as being within the scope of the disclosure, another example of a form for administration would be a solution for injection, in particular for intravenous or intraarterial injection or drip. Usually, a suitable pharmaceutical composition can comprise a buffer (e.g., acetate, phosphate or citrate buffer), a surfactant (e.g., polysorbate), optionally a stabilizer agent (e.g., human albumin), etc. In other methods compatible with the teachings herein, the composition as provided herein can be delivered directly to the organ and/or site of a fibrosis or tumor, thereby increasing the exposure of the diseased tissue to therapeutic agent.

As discussed herein, the composition can be administered in a pharmaceutically effective amount for the in vivo treatment of cancer. In this regard, it will be appreciated that the disclosed composition can be formulated so as to facilitate administration and promote stability of the active agent. Pharmaceutical compositions in accordance with the disclosure can comprise a pharmaceutically acceptable, non-toxic, sterile carrier such as physiological saline, non-toxic buffers, preservatives and the like. Suitable formulations for use in therapeutic methods disclosed herein are described in Remington's Pharmaceutical Sciences (Mack Publishing Co.) 16th ed. (1980).

Certain pharmaceutical compositions provided herein can be orally administered in an acceptable dosage form including, e.g., capsules, tablets, aqueous suspensions or solutions. Such compositions can be prepared as solutions in saline, employing benzyl alcohol or other suitable preservatives, absorption promoters to enhance bioavailability, and/or other conventional solubilizing or dispersing agents.

Methods of Use and Pharmaceutical Compositions

The provided compositions comprising a fusion protein and a STING agonist, wherein the fusion protein comprises STINGΔTM protein fused to a cell-penetrating domain or a nanobody are useful in a variety of applications including, but not limited to, methods of treating and/or ameliorating various diseases and conditions. Methods are provided for the use of the disclosed compositions to treat subjects having a disease or condition associated with STING signaling, altered STING expression. The composition disclosed herein may be used to treat auto-inflammation, virus infection or cancers.

In certain embodiments, the disclosure provides a method of treating cancer that comprises contacting a cancer cell, tumor associated-stromal cell, or endothelial cell with the disclosed composition. In additional embodiments, the cancer cell is a myeloma (e.g., multiple myeloma, plasmacytoma, localized myeloma, or extramedullary myeloma), ovarian, breast, colon, endometrial, liver, kidney, pancreatic, gastric, uterine and/or colon cancer cell. In some embodiments, the contacted cell is from a cancer line. In some embodiments, the cancer cell is contacted in vivo.

Combination Therapies

In some embodiments, the composition comprising a fusion protein and a STING agonist, wherein the fusion protein comprises STINGΔTM protein fused to a cell-penetrating domain or a nanobody is administered alone or as a combination therapy. In some embodiments, composition is administered in combination with one or more other therapies. Such therapies include additional therapeutic agents as well as other medical interventions. Exemplary therapeutic agents that can be administered in combination with the composition provided herein include, but are not limited to, chemotherapeutic agents, and/or immunomodulators. In various embodiments, the composition is administered to a subject before, during, and/or after a surgical excision/removal procedure.

### EXAMPLES

The invention now being generally described, it will be more readily understood by reference to the following examples which are included merely for purposes of illustration of certain aspects and embodiments of the present invention, and are not intended to limit the invention.

### Example 1. Materials and Methods

### Chemicals and Antibodies

Tween-20, Triton X-100, Triton X-114 were all purchased from Sigma-Aldrich (St Louis, Mo.). Carboxyfluorescein succinimidyl ester (CFSE) was purchased from Tonbo Biosciences (San Diego, Calif.). All other chemicals were purchased from ThermoFisher (Waltham, Mass.) and used as received. Human CXCL10/IP-10 and mouse CXCL10/IP-10 ELISA Kit, Murine TNF-alpha, and Murine IFN-gamma were respectively purchased from R&D system (Minneapolis, Minn.) and Peprotech (Rocky Hill, N.J.). Zombie Dyes, Alexa647 anti-DYKDDDDK Tag Antibody (Clone L5), APC anti-mouse CD8a (Clone 53-6.7), FITC anti-mouse CD3 (clone 145-2C11), PerCP-Cy5.5 anti-mouse CD4 (Clone 129.29), PE anti-mouse CD8a (clone 53-6.7), PerCP-Cy5.5 cd11b (Clone M1/70), FITC anti-mouse cd11c (Clone N418), PE anti-mouse CD45 (clone 30-F11), Alexa 488 anti-mouse CD45 (clone 30-F11), FITC anti-human HLA-A,B,C Antibody (clone W6/32), FITC anti-mouse H-2Kb/H-2Db Antibody (Clone 26-8-6) were from Biolegend (San Diego, Calif.). Primary antibodies of STING/TM173 (D2P2F), alpha-Tubulin (DM1A), TBK1/NAK (D1B4) were from Cell signaling technology (CST, Danvers, Mass.). Secondary antibodies of goat anti-rabbit IgG-HRP and goat anti-mouse IgG-HRP are from Santa Cruz Biotech (Santa Cruz, Calif.). InVivoMAb anti-mouse PD-1 (CD279) was purchased from BioXCell (Lebanon, N.H.).

### Expression and Purification of STINGΔTM Protein Variants

The human STINGΔTM protein (139-379aa) and mouse STINGΔTM (138-378aa) variants were synthesized by gblock (IDT, Coralville, Iowa), and cloned into pSH200 vector (from Duke University) containing a 6×histidine tag (His-tag) (SEQ ID NO: 44), between NcoI and NotI sites. Mutants were generated with site-specific mutagenesis based on the human STINGΔTM plasmids. All plasmids were confirmed by sequencing. STINGΔTM variants were expressed as His-tag proteins from BL21 (DE3) Escherichia coli (E. coli). All proteins were expressed as cultures grown in Luria-Bertani broth (LB) (5 g sodium chloride, 5 g tryptone, 2.5 g yeast extract, and 500 mL of distilled water), supplemented with 100 μg/mL Ampicillin. After outgrowth at 37° C. with 225 rpm in a shaker, and until optical density (OD600) reached 0.6, 1 mM IPTG was added to induce the protein expression for 16 to 18 hours at 20° C. and 225 rpm. Cells were then collected by centrifugation at 5000×g for 20 minutes at room temperature. The bacterial pellets were resuspended in a 10 mL protein binding buffer (50 mM sodium phosphate, 0.5 M sodium chloride, 10 mM imidazole) and stored at −80° C. until purification. The frozen cultures were thawed and lysed with 1% Triton-100, 1 mg/mL lysozyme, 1 mM PMSF, and one EDTA-free protease inhibitor cocktail tablet at room temperature for 20 min. The lysate was disrupted by ultrasonication at 5-second intervals for a total of 5 min each at 18 W on ice. Insoluble debris was removed by centrifugation at 12000×g for 60 min, at 4° C. Protein purification was carried out by affinity chromatography using Cobalt agarose beads. 10 mL of raw protein extracts were applied to the protein binding buffer-equilibrated beads, followed by three washes with protein binding buffer plus 0.1% Triton-114 for endotoxin removal. After elution (50 mM sodium phosphate, 0.5 M Sodium chloride, 150 mM imidazole), protein extracts were loaded to fast protein liquid chromatography (FPLC, NGC Quest 10 Chromatography System, Biorad) for 3×PBS buffer exchange and purification. Protein fractions detected at λ=280 nm were collected. Purified STINGΔTM variants concentrations were determined by DC protein assay and purities were verified by SDS-PAGE. Protein aliquots were kept at −80° C. at all times until further use.

### Animal Work

All work with C57BL/6J mice (females, 7-10 weeks old) and OT-1 transgenic mice (The Jackson Laboratory, Bar Harbour, Me.) was performed in accordance with institutional guidelines under protocols of NU-20-0312R (C57BL/6J) and NU-19-0106R (OT-1) approved by Northeastern University-Institutional Animal Care and Use Committee (NU-IACUC). All mice were maintained in a pathogen-free facility following the National Research Council of the National Academies.

### Cell Lines and Cell Culture

Non-small cell lung cancer cell lines A549, H1944, H2122, H23, HCC44 harboring KRAS/LKB1 co-mutations and H1944 Knockouts (H1944 STING-knockout, H1944 cGAS-knockout, H1944 scramble-knockout) were generous gifts from Dr. David Barbie's lab. Human and murine cell lines of B16F10, HeLa, HEK293T, SK-MEL-3, and SK-MEL-5, were obtained from the American Type Culture Collection (ATCC, Rockville, Md.). Yummer1.7 was requested from the Koch Institute (Cambridge, Mass.). B16-OVA(257-264aa) and Yummer1.7-OVA(257-264aa) were generated through transfection with plasmids encoding full lengths of OVA and EGFP, and sorted by FACS for GFP expression. A549, SK-MEL-3, SK-MEL-5, Yummer1.7, HeLa and HEK293T were cultured in Dulbecco's modified Eagle's medium (DMEM) supplemented with 10% fetal bovine serum (FBS), 100 U/mL penicillin-streptomycin, and 100× Non-Essential Amino Acid (NEAA). H1944, H2122, HCC44, and H23 were cultured in RPMI-1640 supplemented with 10% FBS, 100 U/mL penicillin-streptomycin, and 100×NEAA. H1944 STING-knockout, H1944 cGAS-knockout, and H1944 scramble-knockout were cultured in RPMI-1640, with 10% FBS, 100 U/mL penicillin-streptomycin, 100×NEAA with 1 μg/mL puromycin selection. Cells were kept in a humidified incubator with 5% carbon dioxide (CO₂) at 37° C. and routinely tested mycoplasma negative by PCR. All the cell experiments were performed between passages 2 and 10.

### Lentivirus Production and Cell Line Generation

Lentiviral vector plasmids of pFUW Ubc OVA (252-271aa) EGFP, EGFP Luciferase puro (663) were used to generate lentiviral particles. 7.5 μg of packaging plasmid psPAX2, 2.5 μg of envelope plasmid pMD2.G, 10 μg of Lentiviral vector plasmids, and 10 μL TransIT-X2 were mixed in 1 mL Opti-MEM. After 30 minutes of incubation at room temperature, the plasmid mixture was added to 70% confluency HEK293T cells. Supernatants were collected at 48 hours and 72 hours after transfection and centrifuged at 1000 g for 10 minutes to remove the debris. Harvested Lentiviral supernatants were kept at −80 C until further cell line generation. After targeted cell lines of B16F10 and Yummer 1.7 reached 70% confluency, lentiviral supernatants were added to the cells with 8 μg/mL polybrene. Transfected cells were selected with 1 μg/mL puromycin.

### Enzyme-Linked Immunosorbent Assay (ELISA)

For human CXCL10 and mouse CXCL10, cells (1 to 2×10⁴) were cultured with premixed complexes of 40 μg/mL, or 10 μg/mL STINGΔTM variants with or without 1 μg/mL or 0.25 μg/mL cGAMP for 72 hours. Conditioned supernatants were collected for ELISA quantification according to manufacturer's instructions. Values represent the average of four to six replicates from at least two independent experiments. For analysis of anti-OVA IgG level, we conducted the ELISA as previously described. For cytokine quantification in the treatment study, tumors were harvested and grounded in tissue protein extraction reagent (T-PER®) with 1% proteinase inhibitors. The lysates were incubated at 4° C. for 30 min with rotation. The supernatant from each lysate was collected after removing debris through centrifugation. The quantifications of CXCL10, TNF-α, and IFN-γ were performed according to manufacturer's instructions.

### Immunofluorescence Staining

A549, H1944 and HeLa were seeded in chamber slides at a density of ˜5×10⁴ 24 hours before incubation with 40 μg/mL STINGΔTM variants and 1 μg/mL cGAMP complexes. After another 24 hours, cells were washed with PBS once, and fixed with 70% ethanol. After permeabilization with PBS containing 0.1% Triton X-100 for 15 minutes, cells were washed and incubated with the anti-DYKDDDDK Tag antibody (“DYKDDDDK” disclosed as SEQ ID NO: 45) at 1:500 dilution in 1×PBS with 1% BSA and 0.05% TWEEN 20 (PBST) at 4° C. overnight. Cells were then washed for 30 minutes in PBST, and incubated with Alexa488-Phalloidin (CST) in 1:100 dilution for 1 hour. After washing cells with PBST for three times for 10 minutes each, cells were counter-stained with DPAI in mountaing media at room temperature. Images of the cells were visualized and captured by Nikon Eclipse microscope (Tokyo, Japan) and analyzed by ImageJ (NIH).

### Fluorescence Imaging Analysis

Three days after injection with complexes, tumors were harvested and placed in OCT in tissue cassettes and frozen on ice for cutting into 8-10 μm sections in slides. The slides were washed with PBS for 10 min at room temperature, dried on a paper towel and incubated with anti-CD45 diluted in the antibody buffer (10% FBS in PBS) for 1 hour at room temperature in the dark. After three washes with PBS, the slides were fixed in 4% paraformaldehyde in PBS. Slides were incubated with 0.025% saponin in PBS for permeabilization. Anti-DYKDDDDK (“DYKDDDDK” disclosed as SEQ ID NO: 45) were added on the sections for overnight incubation at 4° C. in the dark. Slides were washed in PBS with 0.0025% saponin for 10 min twice. After incubating with secondary antibody for 1 hour in the dark, slides were rinsed with PBS with 0.0025% saponin and counterstained with DAPI. The stained tumor slides were imaged using a Nikon microscope.

### Flow Cytometry

For uptake study, 1×10⁵ cells were seeded in 12-well plates in their corresponding complete culture medium and incubated for 24 hours. After treatment with 40 μg/mL STINGΔTM variants with or without 1 μg/mL cGAMP for 24 hours, cells were washed with PBS and treated with trypsin for at least 15 minutes to remove STING proteins nonspecifically bound to the cell surface. Cells were transferred to 96-well v-bottom plates and collected through 300×g centrifugation for 3 minutes. After twice washes with 200 μL PBS, cells were fixed with 70% ethanol for 20 minutes. The fixed cells were washed with PBS for 10 minutes three times. Cells were resuspended in anti-DYKDDDDK Tag Antibody (“DYKDDDDK” disclosed as SEQ ID NO: 45) at 1:1000 dilution in antibody dilution buffer (1×PBS containing 1% BSA and 0.05% Tween 20) and incubated for 2 hours at room temperature in dark. Antibodies were removed by rinsing cells with PBST three times. The cell suspension in PBS was loaded to Attune flow cytometry (Thermofisher, Waltham, Mass.). Doublets and dead cells were excluded before analysis.

For in vitro MHC-I analysis, 10000 cells were incubated with 40 μg/mL STINGΔTM variants and 1 μg/mL cGAMP in a complete culture medium for 48 hours before staining. Cells were rinsed by PBS, detached by 100 μl 5 mM EDTA in PBS with a fixable live/dead dye, NIR Zombie Dye (Biolegend), at 1:1000 dilution for dead cell exclusion. After staining was quenched by FACS buffer (5% FBS, 2 mM EDTA, 0.1% sodium azide in PBS), cells were resuspended by FACS buffer containing 0.4 μg/mL anti-human HLA-A,B,C antibody or FITC anti-mouse H-2Kb/H-2Db antibody, and incubated on ice for 30 min in dark. Stained cells were washed twice and resuspended in the FACS buffer for flow cytometric analysis in FlowJo (Franklin Lakes, N.J.). After excluding doublets and debris of dead cells, gating strategies determined through control staining were applied for analysis while compared with FITC Mouse IgG2a, κ Isotype Control Antibody stained cells.

For OT-1 CD8+ T cells stimulation, CFSE stained lymphocytes were collected through 500×g centrifuge for 3 min and washed with 200 μl PBS. 100 μl Zombie dye in PBS at 1:1000 dilution was added to the lymphocyte and incubated for 30 min at room temperature avoiding light. Zombie dye staining was quenched by 100 μl FACS buffer. After 3 min centrifuge at 500×g, OT-1 CD8+ T cells were selected by 100 μl APC anti-mouse CD8a Antibody in FACS buffer at 1:1000 dilution after 30 min incubation on ice. Co-stained cells were resuspended in the FACS buffer and quantified under the flow cytometer.

For in vivo tumor profiling, dissected tumors were digested in 1 mg/mL collagenase D for 1 hour at 37° C. Single-cell suspensions were obtained from mincing the tumor through a 70 μm cell strainer. After staining with NIR zombie dye for dead cell exclusion, cells were neutralized and blocked with anti-CD16/CD32 for 5 minutes on ice and stained with antibodies against surface markers CD45, CD3, CD4, CD8, CD11b, CD11c on ice for 30 minutes in FACS buffer. For intracellular staining, cells were fixed, permeabilized, and stained with anti-DYKDDDDK tag antibody (“DYKDDDDK” disclosed as SEQ ID NO: 45). All samples were analyzed by FlowJo after loading to the flow cytometer.

### Cell Viability Assay

The effects of STINGΔTM variants and cGAMP complexes on cell viability were determined by 3-(4,5-dimethylthiazol-2-yl)-2,5-diphenyltetrazolium bromide (MTT) assay. 1000 cells were seeded in 96-well plates and treated with 40 μg/mL STINGΔTM variants and 1 μg/mL cGAMP, for 120 hours in 5% C02 at 37° C. in a humidified incubator. Cells were further incubated with 0.5 mg/mL MTT dissolved in sterilized 1×PBS at 37° C. for 2 hours before DMSO was added into each well to dissolve formazan crystals. The absorbance of each well was determined at 570 nm on an automated Bio-Rad microplate reader (Bio-Rad Laboratories, Hercules, Calif.). Untreated cells as control were considered to be 100% viable.

Lymphocyte Preparation from Lymph Nodes in OT-1 Mice

The mesenteric, inguinal, axillary, and brachial lymph nodes dissected from OT-1 mouse were homogenized to generate a single cell suspension, and the released cells in lymphocyte growth medium (RPMI1640 complete media and 50 μM 2-mercaptoethanol) were pelleted and resuspended in 10 ml PBS. The lymphocyte was washed and stained with 1 μM CFSE in 1×PBS for 20 min until the staining was terminated by 10% FBS. The stained lymphocyte was resuspended and cultured in lymphocyte growth medium in a humidified incubator to release excessive CFSE. After 2 hours incubation, lymphocyte was collected and resuspended in lymphocyte growth medium with 20 U/mL interleukin (IL)-2.

Coculture of OT1 Lymphocytes with B16-OVA or YUMMER 1.7-OVA

100 μl of 1×10⁶ lymphocytes in lymphocyte growth medium with 20 U/mL IL-2 was added into the 96-well plate with 100 μl of 1×10⁴ B16-OVA(257-264aa) treated with STINGΔTM variants with or without cGAMP 48 hours ahead. On days 3, 100 μl of lymphocytes were gently collected for flow cytometry analysis. 100 μl fresh lymphocyte growth medium with 20 U/mL IL-2 was added to each well for leftover lymphocyte growth. On day 5, after lymphocytes were collected, B16-OVA(257-264aa) attached wells were washed with PBS twice for subsequent MTT assay.

### Immunizations, Tumor Inoculation and Treatment in Mice

Analysis of immunizations for adjuvant potential performed in C56BL/6 mice with B16-OVA (257-264aa) was conducted as previously described. For treatment study, one million Yummer1.7 cells in 100 μl Opti-MEM were subcutaneously injected into the flank of mice. At 6-9 days later when tumors reached 100 mm\hat{ }3 in volume, animals were injected intratumorally with ˜25 ul vehicle control, 2.5 μg cGAMP only or 100 μg STINGΔTM variants and 2.5 μg cGAMP complex in Opti-MEM.

### Statistical Analysis

Statistical significance was evaluated using one-way ANOVA followed by Tukey post hoc test. P values less than 0.05 were considered significant. Statistical significance is indicated in all figures according to the following scale: *P<0.05, **P<0.01, ***P<0.001, and ****P<0.0001. All graphs are expressed as the means±SEM. In one-way ANOVA followed by post hoc tests, we marked asterisks only in pairs of our interest.

### Example 2. Overall Scheme of cGAMP Delivery by CP-STINGΔTM

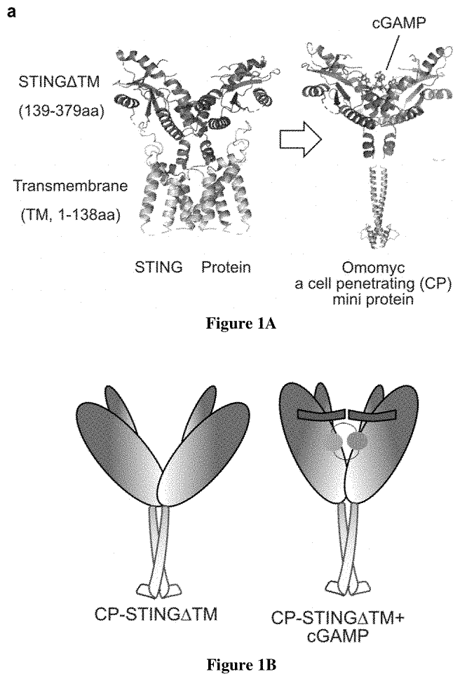

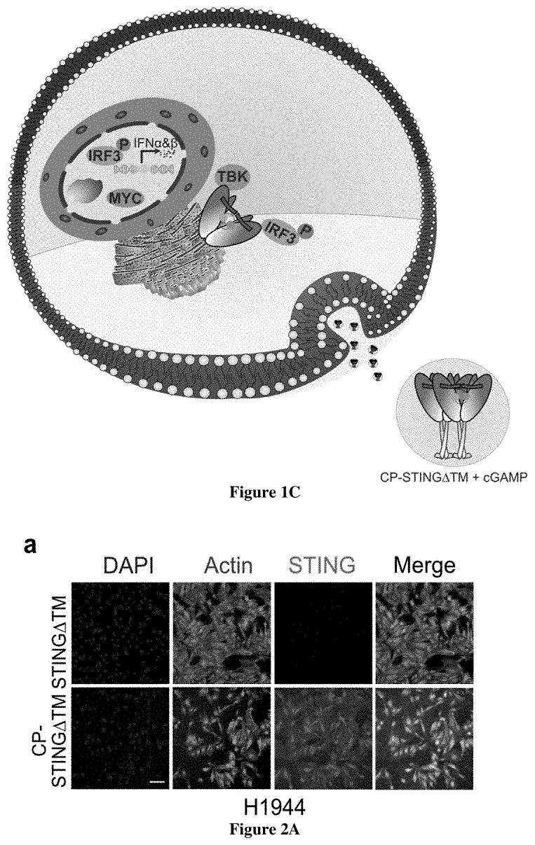

In contrast to existing delivery strategies such as nanoformulations or synthetic depots to overcome the challenges in encapsulation and intracellular delivery of STING agonist (e.g. cGAMP), we have repurposed the natural receptor STING as a highly modular and simple platform to efficiently bind and deliver cGAMP in vitro and in vivo. Specifically, the recombinant C-terminal domain of STING protein (STINGΔTM, 139-379aa for human and 138-378aa for mouse) binds cGAMP with high affinity and stability. Additionally, we uncovered that while the recombinant STINGΔTM purified from E. coli lacks the N-terminal transmembrane domain that is crucial for the oligomerization and translocation of the endogenous full-length STING from the endoplasmic reticulum (ER) to the Golgi apparatus, the recombinant STINGΔTM could form complexes with cGAMP, and activate the downstream STING signaling following delivery of the complexes by commercial transfection reagents in HEK293T that do not express endogenous STING. To the contrary, recombinant STINGΔTM proteins with mutations including S366A and deletion of last 9 amino acids (i.e. ΔC9), which are known to abolish the engagement of STING with downstream effector proteins such as TBK1, failed to activate the STING pathway in HEK293T. The findings in STING-negative cells confirmed that recombinant STINGΔTM protein exhibits distinct function as endogenously expressed STINGΔTM lacks the capability of inducing the type I IFN. Building on our earlier discovery, to bypass the need for transfection reagents, here we developed a cell-penetrating (CP)-STINGΔTM to deliver cGAMP into different cell types via genetic fusion of a cell-penetrating protein (FIGS. 1A and 1B). Notably, in contrast to cell-penetrating peptides such as trans-activating transcriptional activator (TAT), we have chosen the Omomyc mini-protein as our cell-penetrating moiety for three reasons: (1) Omomyc (91 amino acids) is derived from a dominant-negative form of the human MYC oncogene and has recently shown specific targeting and potent tumor cell penetration capabilities in human cancer cell lines and xenograft mouse models; (2) The natural dimer conformation of Omomyc coincides with STINGΔTM, which also exists as a dimer in the absence of cGAMP; (3) Omomyc may not cause an immunogenicity issue owing to its human origin. Other cell-penetrating peptides including TAT are also used.

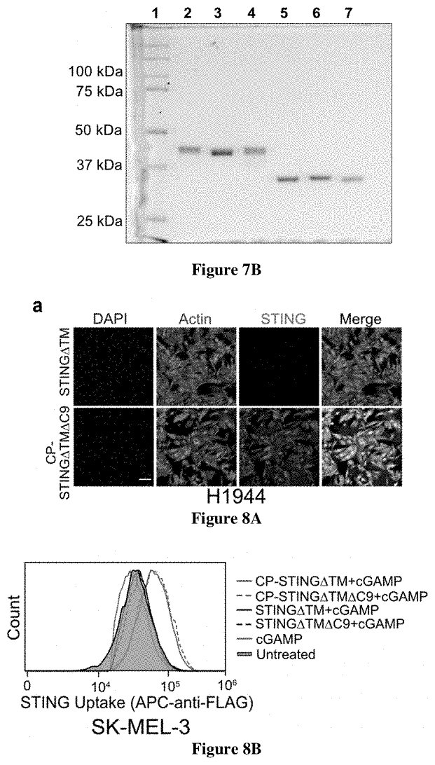

Since the C terminal amino acids of STING directly interact with downstream effector proteins including TBK1 and IRF3, we genetically fused the cell-penetrating protein Omomyc to the N terminus of STINGΔTM to prevent any steric hindrance posed by Omomyc (FIG. 1C). In addition, we generated two essential CP-STINGΔTM mutants to help dissect the mechanisms underlying enhanced delivery of cGAMP: one lacks the effector function to engage with the downstream STING signaling pathway and the other fails to bind cGAMP (Table 1). After recombinant protein expression in E. coli, we purified 6×Histidine (SEQ ID NO: 44) (His) tagged proteins via the metal affinity purification and size exclusion chromatography. As shown in FIG. 7, both size exclusion chromatography studies and SDS-PAGE confirm that the fusion protein can be purified with high yield and homogeneity from E. coli. Additionally, the denatured proteins exhibited predicted molecular weights in SDS-PAGE, while the SEC graphs show that CP-STINGΔTM likely forms a tetramer under a native condition in agreement with our previous study.

TABLE 1
STING variants used in this study. *Amino acid positions represent
the human STING (1-379aa), which are conserved
in the mouse STING (1-378aa).

| STING variants* | Description |
|---|---|
| STINGΔTM | STING lacking the N terminal transmembrane |
| | domain |
| STINGΔTMΔC9 | 9-amino acid deletion at the C terminus that |
| | abolishes type 1 IFN induction |
| STINGΔTM(R238A/ | Deficient for cGAMP binding |
| Y240A) | |
| CP-STINGΔTM | Inclusion of cell-penetrating |
| | domain—Omomyc to bypass |
| CP-STINGΔTMΔC9 | transfection reagent |
| CP- | |
| STINGΔTM(R238A/ | |
| Y240A) | |
| CP-STINGΔTM-dsred | |

### Example 3. CP-STINGΔTM can Effectively Internalize Cells

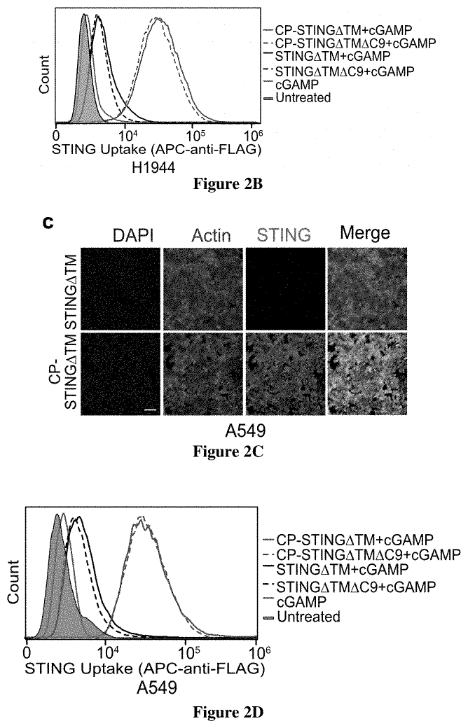

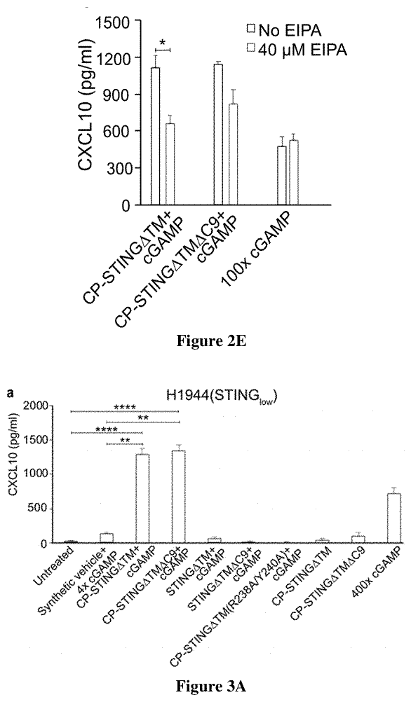

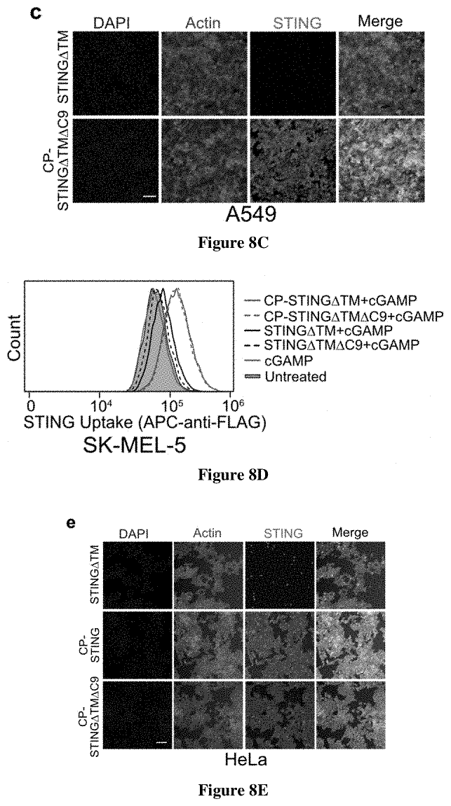

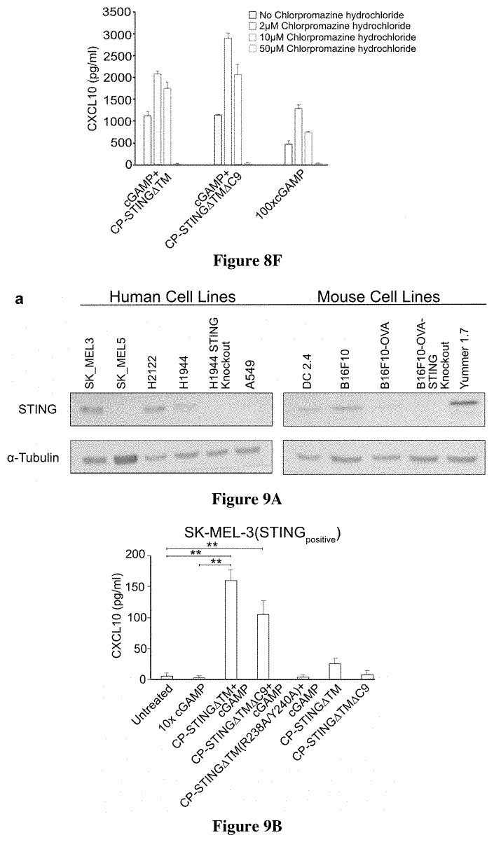

Despite the Omomyc protein itself has been shown to internalize different lung cancer cell lines in vitro as well as in mouse lung xenografts, it remains to be investigated whether genetic fusion of Omomyc with STINGΔTM can indeed penetrate cells spontaneously. To assess the cell-penetrating potential of CP-STINGΔTM, we treated two human non-small cell lung cancer (NSCLC) cell lines, H1944 and A549 for 24 hours, followed by immunostaining against an 8-amino acid epitope (DYKDDDDK (SEQ ID NO: 45)), named FLAG, which is encoded in between Omomyc and STINGΔTM. Because the FLAG epitope is not known to be expressed by mammalian cells, we could make use of anti-FLAG staining to distinguish exogenously delivered STING protein variants from endogenous STING proteins. Moreover, in contrast to covalently conjugating proteins with fluorescent dyes, which typically modify the surface amine or cysteine groups of proteins, our approach can prevent altering the pharmacokinetics of intracellular protein accumulation. As shown in FIGS. 2A and 2C, CP-STINGΔTM exhibited efficient intracellular uptake in H1944 and A549, while STINGΔTM alone failed to internalize cells owing to the lack of Omomyc to promote cell penetration. In addition, we also genetically fused Omomyc to the catalytically inactive mutant STINGΔTMΔC9, which is known to abolish the STING function due to the deletion of 9 amino acids at the very C terminus. As shown in FIGS. 8A, 8C, and 8E, the CP-STINGΔTMΔC9 showed comparable degrees of internalization, which confirmed that the intracellular uptake is mediated by Omomyc instead of STING. To further corroborate our findings in fluorescence microscopy, we performed flow cytometry to confirm the uptake profiles of different STING variants after intracellular staining against the same synthetic epitope FLAG (FIGS. 2B and 2D). In addition to the NSCLC cell lines, we validated the uptake of CP-STINGΔTM and CP-STINGΔTMΔC9 in human melanoma and ovarian cancer cell lines by fluorescence microscopy and flow cytometry (FIGS. 8B, 8D and 8E). Finally, to dissect the mechanism by which the cell-penetrating STINGΔTM enters cells, we tested a range of small molecule inhibitors targeting different endocytic pathways including: 5-(N-Ethyl-N-isopropyl) amiloride (EIPA), chlorpromazine, Dynasore, cyclodextrin, and Filipin. Among the small molecule inhibitors we have tested, a macropinocytosis inhibitor, EIPA exhibited a dose-dependent inhibition of cell-penetrating STINGΔTM in H1944 (FIG. 2E). In contrast, inhibitors targeting other uptake pathways failed to inhibit the uptake of cell-penetrating STINGΔTM (FIG. 8C). The Omomyc protein itself was taken up by cancer cells primarily through macropinocytosis. Therefore, we conclude that the cell-penetrating capability of the fusion protein is mediated by Omomyc in a macropinocytosis-dependent manner.

### Example 4. CP-STINGΔTM can Markedly Enhance cGAMP Delivery and STING Activation In Vitro

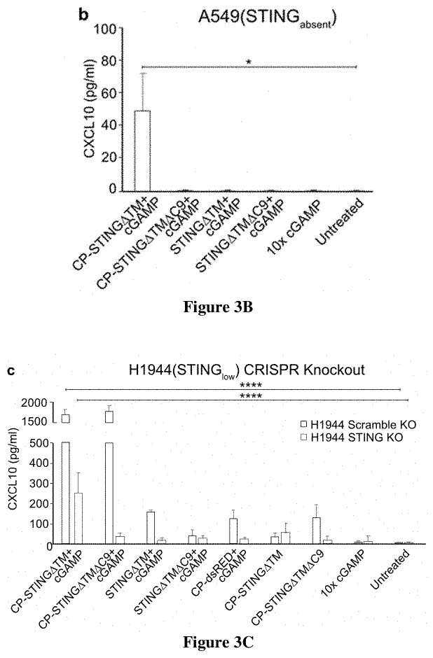

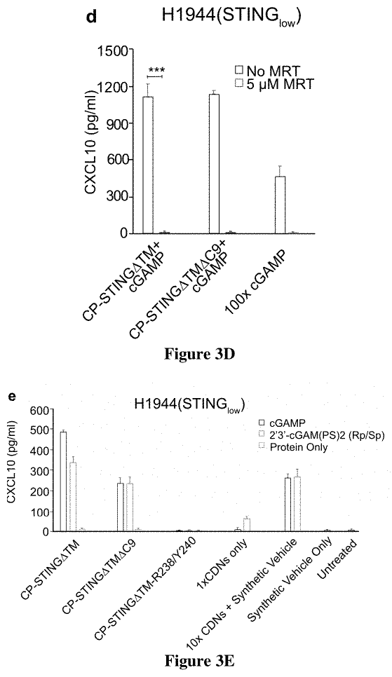

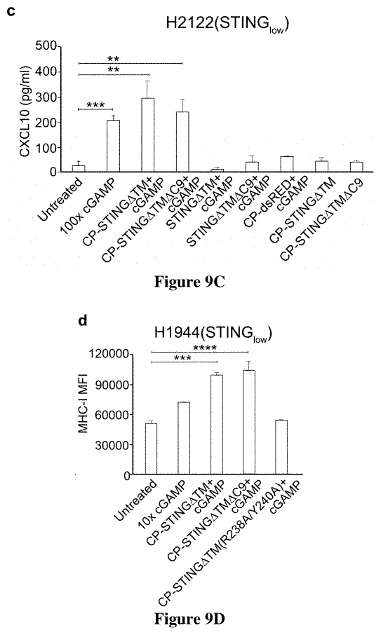

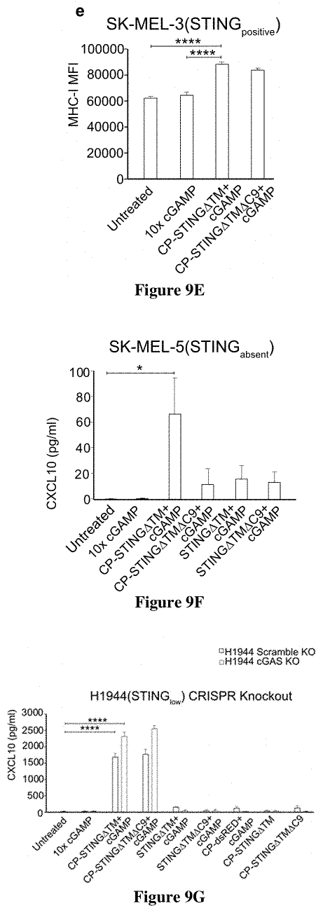

In contrast to innate immune cells, which are highly sensitive to cGAMP-mediated STING activation, previous work by others have shown that downregulation of STING in tumor cells greatly reduced the sensitivity of cancer cells to STING agonists, which can promote immune suppression and exclusion of cytotoxic T cells in the tumor microenvironment. Therefore, we sought to ask whether the fusion protein could promote intracellular delivery of the STING agonist cGAMP in a panel of cell lines with reduced sensitivity to STING agonists. We first focused on two NSCLC cell lines, H1944 and H2122 (STING_{Low}), of which the expression of endogenous STING is downregulated due to histone methylation at the native STING promoter. As shown in FIGS. 3A and 9C, we compared CP-STINGΔTM+cGAMP, CP-STINGΔTMΔC9+cGAMP, free cGAMP and lipofectamine-transfected cGAMP to vehicle control-treated cells. Of note, a 1:1 molar ratio of one STING dimer to one cGAMP was prepared for different STING/cGAMP complexes. Impressively, the co-delivery systems comprising CP-STINGΔTM+cGAMP or CP-STINGΔTMΔC9+cGAMP, required ˜100-fold lower concentration of cGAMP than free cGAMP or lipofectamine-transfected cGAMP to induce comparable levels of CXCL10, one of the chemokines that can be induced by the STING pathway. In addition, since the STING activation in tumor cells can upregulate major histocompatibility complex I (MHC-I) to promote cytotoxic T cell recognition, we measured the surface expression of MHC-I in the same cancer cells. Consistent with measurement of CXCL10 by ELISA, CP-mSTINGΔTM+cGAMP and CP-mSTINGΔTMΔC9+cGAMP similarly enhanced surface expression of MHC class I in H1944 and melanoma cells (FIGS. 9D and 9E).

To explain our findings, we first ruled out the possibility of endotoxin contamination resulting from protein purification from E. coli, as CP-STINGΔTM or CP-STINGΔTMΔC9 protein alone of equivalent concentrations did not induce CXCL10 (FIG. 3A). It is intriguing, however, delivery of cGAMP by the catalytically inactive CPSTINGΔTMΔC9, in which the interaction of STING with TBK1 and IRF3 is disabled, enhanced the STING activation to a degree similar to that of the wildtype (i.e. CP-STINGΔTM) (FIG. 3A). We hypothesized that in the STING_{Low} cell lines H1944 and H2122, the cell-penetrating-STINGΔTM primarily may serve as a chaperon by promoting delivery of cGAMP into tumor cells. To test this hypothesis, we generated two additional fusion proteins: CP-dsRed and CP-STINGΔTM (R238A/R240A). Importantly mutations of the 238th arginine (R238) and 240th tyrosine (Y240) to alanine (A) are known to abolish the ability of STING to bind cGAMP. As shown in FIGS. 3A, 9D and 9E, these two protein variants failed to enhance CXCL10 production to the same extent as CP-STINGΔTM+cGAMP and CP-STINGΔTMΔC9+cGAMP. Therefore, through genetic mutations that inactivate two separate functions of STING including the effector and cGAMP-binding capabilities, we have found that in STING_{Low} cells, CP-STINGΔTM primarily act as a chaperon to efficiently deliver cGAMP intracellularly and therefore greatly enhancing the STING activation.

Motivated by the ability of CP-STINGΔTM to markedly enhance cGAMP delivery and STING activation in STING_{Low} cells, we further extended our observations to A549 (human NSCLC) and SK-MEL-5 (human melanoma), which do not express endogenous STING (STING_{absent}). Interestingly, we found that only CP-STINGΔTM+cGAMP induced CXCL10, while the catalytically inactive CP-STINGΔTMΔC9 along with cGAMP did not (FIG. 3B). Additionally, STINGΔTM+cGAMP failed to induce CXCL10, which can be explained by the absence of Omomyc to facilitate cell penetration (FIG. 3A). These observations imply that codelivery of CP-STINGΔTM and cGAMP functionally restored the deficient STING signaling in STING_{absent} cells. To further confirm this hypothesis, we utilized Clustered Regularly Interspaced Short Palindromic Repeats (CRISPR) to genetically knock out endogenous cGAS and STING, respectively in H1944. Notably, the cGAS knockout is known to inhibit the production of endogenous cGAMP. Consistent with data in STING_{low} cell lines, in H1944 with cGAS knockout but intact STING, both CP-STINGΔTM+cGAMP and CP-STINGΔTMΔC9+cGAMP could comparably induce CXCL10 expression, suggesting that endogenous cGAMP is not required for the activation of STING signaling (FIG. 9F). In H1944 with only STING knockout, however, CXCL10 expression was induced by CP-STINGΔTM+cGAMP but not the catalytically inactive CP-STINGΔTMΔC9+cGAMP (FIG. 3C), which is consistent with findings in A549 and SK-MEL-5 cells, in which endogenous STING expression is completely absent (FIGS. 3B and 9F). In addition, concurrent treatment with a TBK1 inhibitor, MRT, failed to enhance the production of CXCL10 in the cells treated with CP-STINGΔTM+cGAMP and CP-STINGΔTMΔC9+cGAMP (FIG. 3D). Therefore, through both genetic and pharmacological inhibition targeting key protein components in the STING pathway, we have shown that CP-STINGΔTM+cGAMP acts as a functional complex to induce STING signaling in the cells lacking endogenous STING expression. Finally, since cGAMP can be degraded by Ectonucleotide pyrophosphatase/phosphodiesterase 1 (ENPP1), which is abundant in extracellular and intracellular environments, another possibility for enhanced cGAMP delivery is that CP-STINGΔTM may protect cGAMP from ENPP1-mediated hydrolysis. To test this possibility, we explored cGAM(PS)₂(Rp/Sp), a synthetic nondegradable cGAMP analog, in H1944, and observed that CP-STINGΔTM+cGAM(PS)₂(Rp/Sp), and CP-STINGΔTMΔC9+cGAM(PS)₂(Rp/Sp), markedly enhanced CXCL10 production in comparison to cGAM(PS)₂(Rp/Sp) alone of equivalent concentration or at a 10× concentration transfected by a commercial transfection reagent. Moreover, CP-STINGΔTM (R238A/R240A), in which the two mutations R238A and R240A abolish the cGAMP binding, failed to induce CXCL10 in the codelivery with cGAM(PS)₂(Rp/Sp) (FIG. 3E).

### Example 5. Cell Penetrating STINGΔTM Enhanced the Efficacy of cGAMP as an Immune Adjuvant

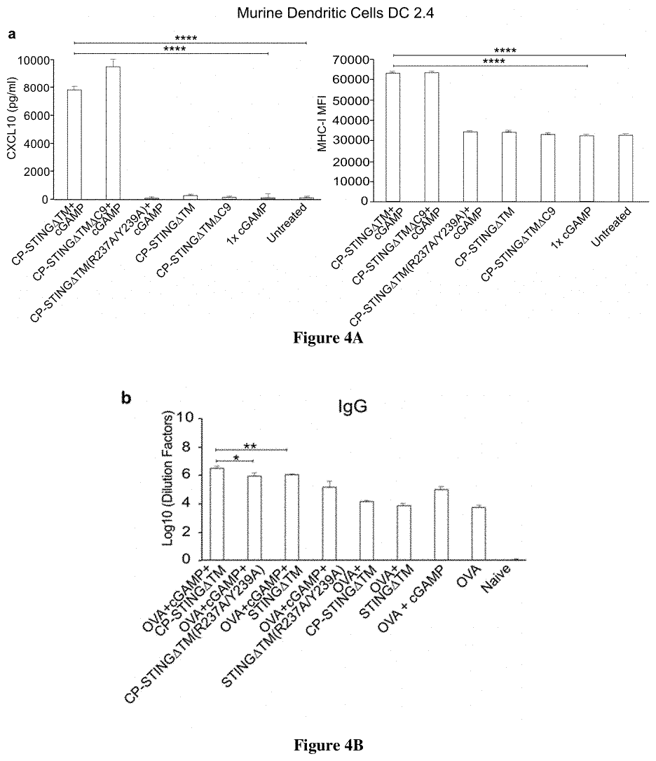

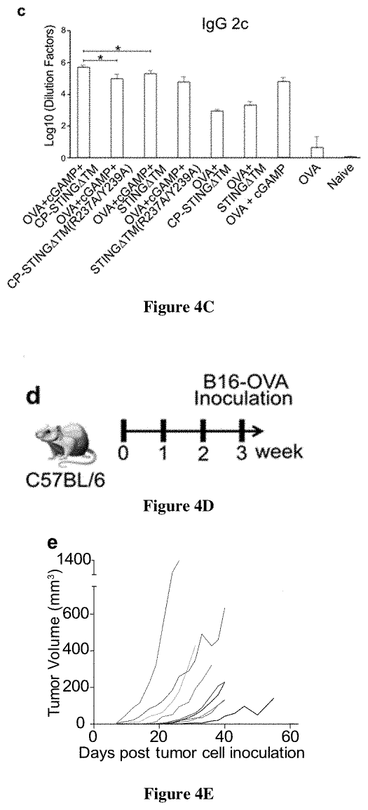

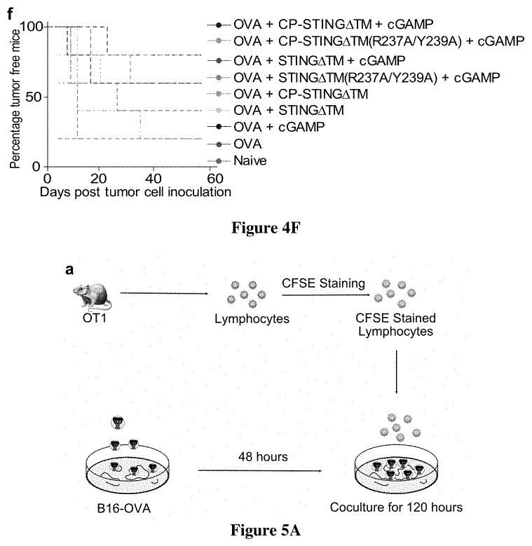

cGAMP has been explored as a potent vaccine adjuvant that promotes both humoral and cellular immune responses in different mouse vaccination models. However, free cGAMP is prone to fast clearance and degradation owing to low molecular weight (˜600 Da) and the presence of hydrolyzable phosphoester bonds respectively. To address these limitations, a myriad of synthetic biomaterials have been developed to enhance the delivery efficacy of cGAMP. In our own work, motivated by enhanced activation of the STING pathway by CP-STINGΔTM in different cell types, we ask whether it could serve as a protein-based delivery platform to efficiently deliver cGAMP as an immune adjuvant. To this end, we made use of the murine dendritic cell line DC 2.4 as a model of antigen presenting cells (APCs). Similar to our findings in cancer cells, it was shown that CP-STINGΔTM+cGAMP greatly induced expression of CXCL10 by ELISA and surface expression of MHC-I compared to free cGAMP as evidenced by flow cytometry (FIGS. 4A and 4B).

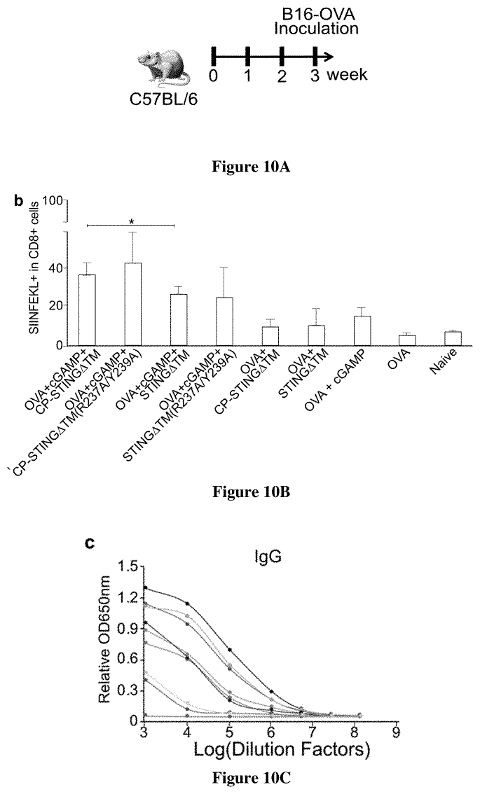

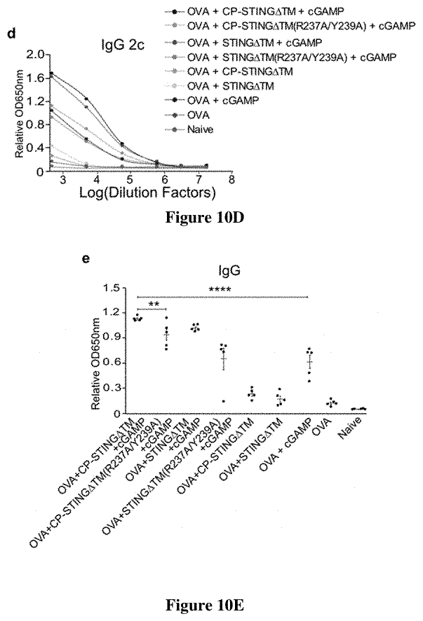

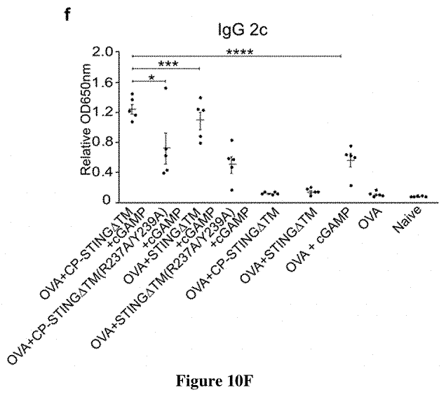

Next, we tested our hypothesis in wild-type C57BL/6 mice by vaccinating them with a model antigen, chicken ovalbumin (OVA), along with free cGAMP or cGAMP+CP-STINGΔTM serving as an immune adjuvant. Following a priming-boost protocol with a two-week interval, we quantified the levels of OVA-specific total IgG as well as type I IFN-associated IgG2c from mouse serum, of which the latter IgG subtype can be induced by the STING activation. As shown by the OVA-specific ELISA, the “OVA+cGAMP+CP-STINGΔTM” treatment group induced ˜10-fold improvement in the levels of OVA-specific IgG and IgG2c as compared with “OVA+cGAMP+CP-STINGΔTM (R237A/Y239A)”, “OVA+cGAMP+STINGΔTM”, and “OVA+cGAMP” (FIGS. 4B, 4C, and 10A-10D). Of note, CP-STINGΔTM (R237A/Y239A) bear mutations that abolish cGAMP binding while STINGΔTM lacks the cell-penetrating domain Omomyc. To examine the cellular responses, we measured the percentage of CD8 T cells carrying the MHC-I-SIINFEKL epitope (“SIINFEKL” disclosed as SEQ ID NO: 43) from OVA₂₅₇₋₂₆₄ₐₐ via tetramer staining (FIG. 10B). In agreement with studies in humoral responses, “OVA+cGAMP+CP-STINGΔTM” increased the induction of SIINFEKL-specific CD8 T cells (“SIINFEKL” disclosed as SEQ ID NO: 43) among different treatment groups. Notably, when we mutated two amino acids in CP-STINGΔTM, (i.e. R237A/Y239A), which are known to abolish the ability of binding cGAMP, no significant reduction in both humoral and cellular immune responses were detected owing to potential non-specific binding of cGAMP. This observation agrees with our studies in cells expressing endogenous STING, where CP-STINGΔTM serves as a chaperon to enhance the cGAMP delivery as opposed to relying on its effector function to engage with downstream targets.

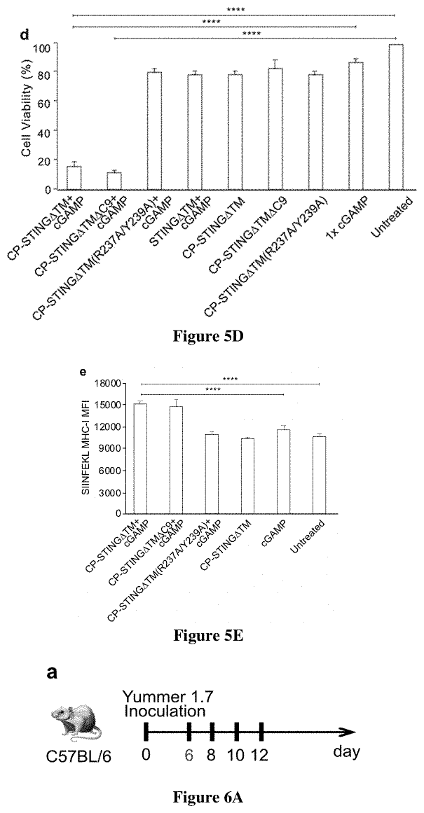

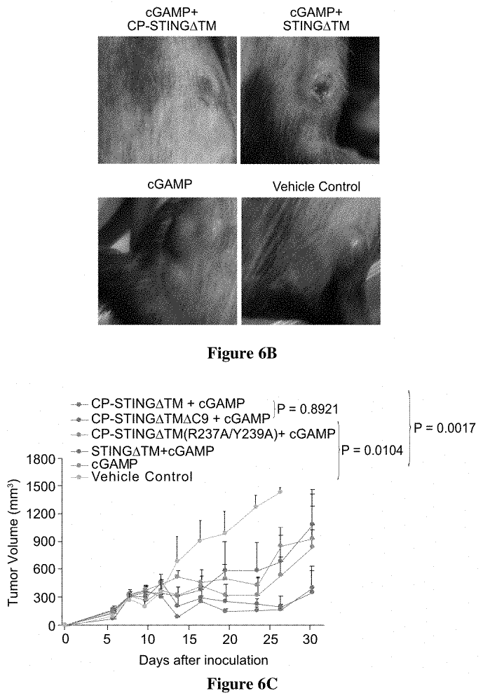

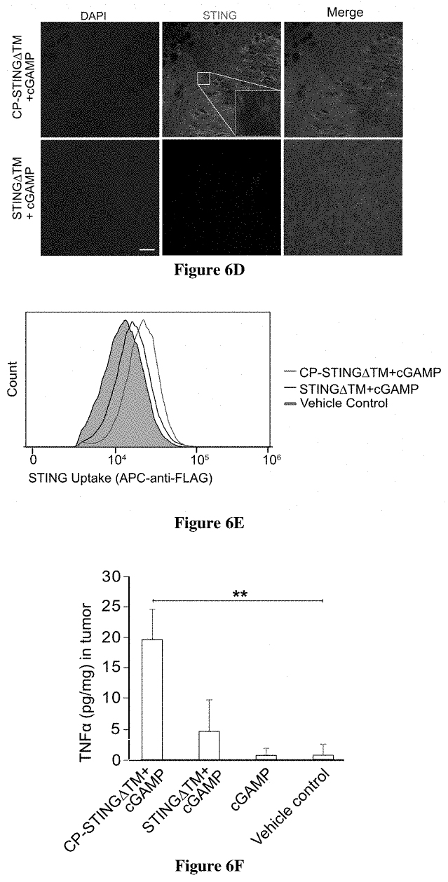

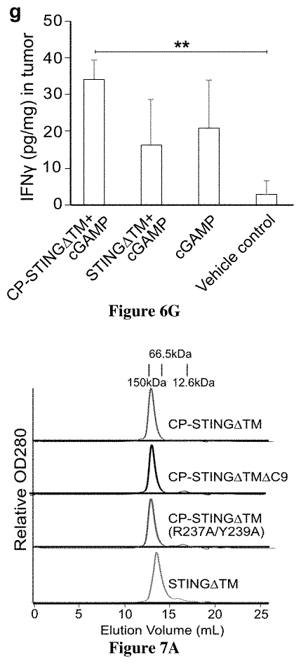

Furthermore, when comparing the CP-STINGΔTM to STINGΔTM alone, the latter of which does not have the cell penetrating protein domain, CP-STINGΔTM markedly enhanced OVA-specific IgG and IgG2c as well as SIINFEKL-restricted CD8 T cells (“SIINFEKL” disclosed as SEQ ID NO: 43). We reasoned that it is due to increased retention and intracellular uptake mediated by the cell penetrating protein Omomyc since in a separate experiment we found that CP-STINGΔTM exhibited greater retention in tumors than STINGΔTM (FIGS. 6D and 6E). Next, we made use of the same cohort of vaccinated C57BL/6 mice to examine whether the increased induction in antigen-specific IgG and CD8 levels could confer a greater protection in a prophylactic syngeneic mouse melanoma model. Specifically, one week after the boost, we challenged the mice with B16 melanoma cells engineered to express the SIINFEKL epitope (SEQ ID NO: 43). As shown in FIGS. 4E and 4F, the cohort vaccinated with OVA+cGAMP+CP-STINGΔTM combination displayed the slowest tumor growth rates and longest survival rates.

### Example 6. Codelivery of CP-STINGΔTM and cGAMP Enhance Tumor Cell Killing by Antigen-Specific T Cells Ex Vivo

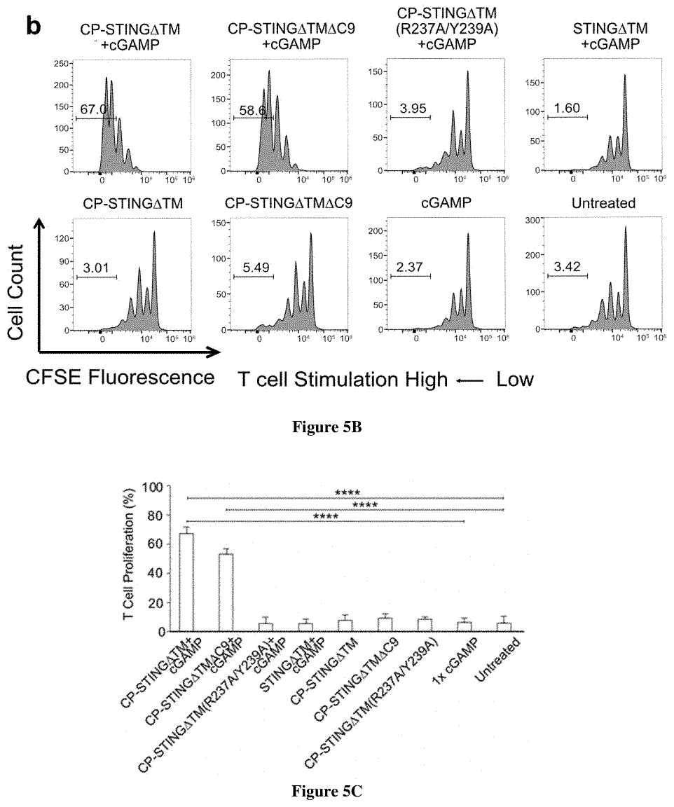

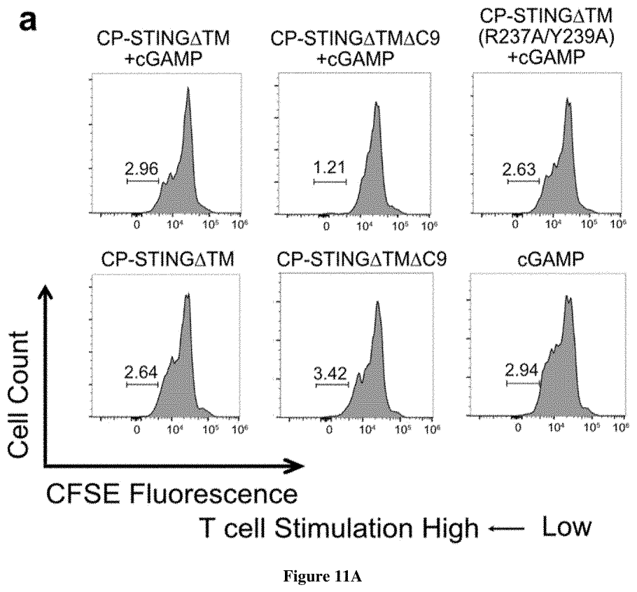

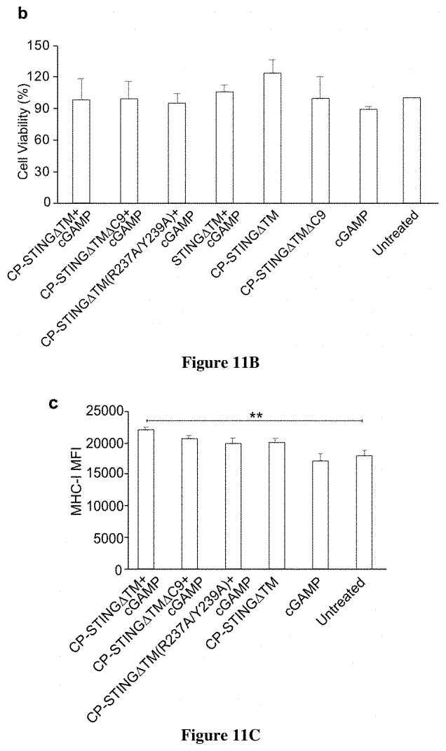

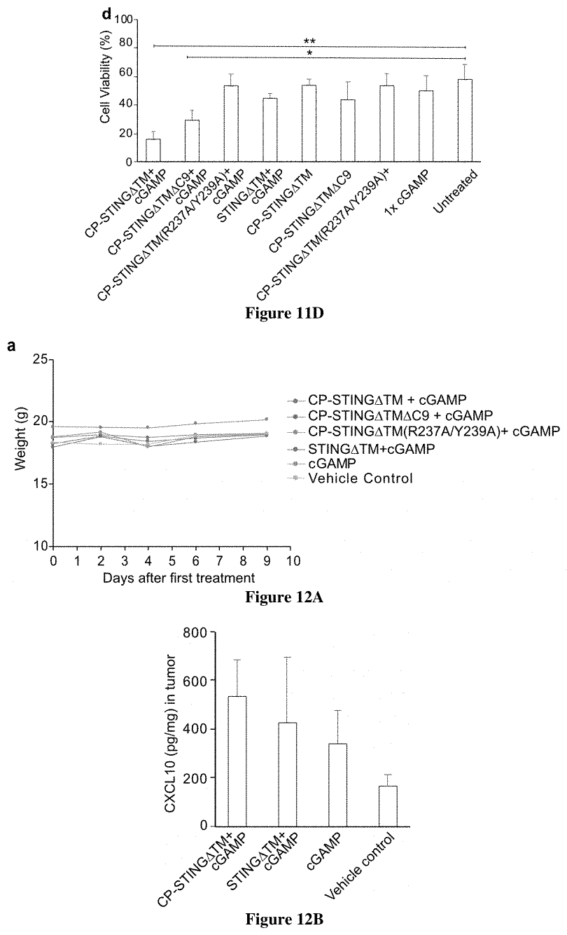

In addition to promoting maturation and cross presentation of dendritic cells for T cell priming, which serves as the very first step of immune clearance of tumor cells, activation of the STING pathway in tumor cells has been shown to augment cytotoxic T cell-mediated cancer cell killing by upregulating MHC-I on the surface of tumor cells. Motivated by the above vaccination and prophylactic cancer models, we sought to test whether CP-STINGΔTM and cGAMP can enhance tumor cell killing. To this end, in an ex vivo model, we generated two isogenic B16 melanoma cell lines expressing either SIINFEKL-GFP fusion (“SIINFEKL” disclosed as SEQ ID NO: 43) or GFP alone, and treated them with free cGAMP, cGAMP+CP-STINGΔTM, cGAMP+CP-STINGΔTMΔC9 and cGAMP+CP-STINGΔTM (R237Y239A) for 48 hr. After the supernatant was removed from the tumor cells, CFSE-stained SIINFEKL-specific CD8 T cells (“SIINFEKL” disclosed as SEQ ID NO: 43), which were harvested from lymph nodes of OT-1 mice, were co-cultured with tumor cells (FIG. 5A). It is noteworthy that by pretreating tumor cells with cGAMP and different STING protein variants followed by washing and co-culturing with antigen specific T cells, we specifically tested the effects of STING activation in tumor cells. As shown in FIGS. 5B and 5S, following a 120 hr coculture, cGAMP complexed with CP-STINGΔTM and CP-STINGΔTMΔC9 induced highest T cell proliferation as evidenced by T cell division-mediated CFSE dilution in flow cytometry. Moreover, the highest efficacy of tumor killing was detected in the same treatment groups by staining viable tumor cells with MTT after washing away nonadherent T cells (FIG. 5D). Of note, the tumor killing was only detectable in B16 cells bearing the SIINFEKL epitope (SEQ ID NO: 43) but not in the GFP-expressing B16 cells in the coculture with OT-1 cells, indicating that the increased T cell proliferation and tumor cell killing were antigen-specific (FIGS. 11A and 11B). To confirm that the increased T cell proliferation and killing is due to the enhanced recognition of tumor cells, after treating SIINFEKL-expressing B16 (“SIINFEKL” disclosed as SEQ ID NO: 43) with cGAMP and different STING variants for 48 hr, we quantified the expression levels of MHC-I and SIINFEKL-restricted MHC-I (“SIINFEKL” disclosed as SEQ ID NO: 43) on the surface of tumor cells by flow cytometry. As shown in FIGS. 5E and 11C, only CP-STINGΔTM+cGAMP and CP-STINGΔTMΔC9+cGAMP markedly upregulate the expression of MHC-I and SIINFEKL-restricted MHC-I (“SIINFEKL” disclosed as SEQ ID NO: 43) in comparison to free cGAMP and other control treatment groups. We reason that since B16 cells express endogenous STING (FIG. 9A), CP-STINGΔTM acted as a chaperon to enhance cGAMP delivery into tumor cells.

### Example 7. Codelivery of CP-STINGΔTM and cGAMP Enhances the Therapeutic Efficacy of Immune Checkpoint Blockade

Motivated by enhanced immune stimulation mediated by codelivery of CP-STINGΔTM and cGAMP in the ex vivo tumor cell killing by OT-1 cells, we further examined whether this approach could augment the efficacy of the combination immunotherapy involving STING agonism and immune checkpoint blockade (ICB). Here, we made use of an immunogenic mouse melanoma cancer model bearing YUMMER1.7 tumor cells for three reasons: First, YUMMER1.7 cells carru Braf mutation and Pten loss that mimic the most frequent mutations happening in melanoma patients. Second, tumors with increased immunogenicity are generally responsive to ICB such as anti-PD-(L)1, among which lung cancer and melanoma are of high mutation burden. Third, STING activation in the tumor microenvironment (TME) has been shown to improve the therapeutic efficacy of ICB in different syngeneic mouse cancer models.

Before the treatment study, we first confirmed that CP-STINGΔTM can internalize tumor cells and other cell types in the TME. Specifically, when YUMMER1.7 tumors reached ˜150 mm³ in C57BL/6 mice, a single dose of CP-STINGΔTM was administered intratumorally. Mice were sacrificed at 96 hr, and tumors were harvested for cryo-sectioning and immunostaining using the anti-FLAG antibody specific for recombinant STING protein variants. As shown in FIG. 6D, CP-STINGΔTM was readily detectable across different areas of tumor slices in a homogeneous pattern even at 96 hr after a single intratumoral administration. In contrast, STINGΔTM did not have noticeable signal, suggesting that the presence of the cell penetrating domain Omomyc domain facilitates the retention of recombinant STING in the TME. To corroborate this finding, in a separate cohort of mice, single cells were prepared for intracellular staining against the same FLAG epitope. Similar to our in vitro cellular uptake studies, CP-STINGΔTM efficiently internalized tumor cells in comparison to STINGΔTM that lacks the cell-penetrating capability (FIGS. 6D and 6E).

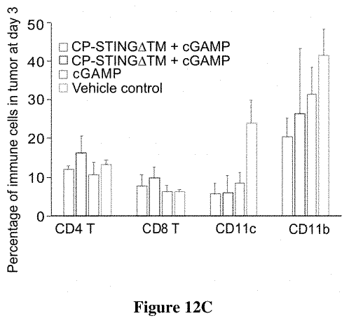

Next, we investigated the therapeutic efficacy of CP-STINGΔTM and cGAMP in combination with anti-PD1 in the Yummer 1.7 syngeneic mouse model. Of note, we initiated treatment in mice with relatively large subcutaneous tumors, which are more challenging to treat with immunotherapy than smaller tumors. After tumors reached ˜150 mm³, CP-STINGΔTM, CP-STINGΔTMΔC9, CP-STINGΔTM(R237A/Y239A) and STINGΔTM were intratumorally administered with cGAMP, while anti-PD1 was given intraperitoneally at optimized doses every two days for a total of four treatments (FIG. 6A). Over the duration of treatment, no significant weight loss was detected among different treatment groups in comparison to the vehicle control group (FIG. 12A). Importantly, both CP-STINGΔTM and CP-STINGΔTMΔC9 showed marked reduction in the tumor progression compared to CP-STINGΔTM(R237A/Y239A) and STINGΔTM treatment groups (FIGS. 6B and 6C). These findings agree with our studies in vitro: (1) The mutations R237A/Y239A in STING abolish the binding of cGAMP, and therefore CP-STINGΔTM(R237A/Y239A) cannot effectively deliver cGAMP into target cells. (2) STINGΔTM alone cannot efficiently penetrate target cells due to the absence of the Omomyc protein. (3) Because cancer cells and hematopoietic cells in tumors express endogenous STING, CP-STINGΔTM plays a chaperon role in enhancing the intracellular delivery of cGAMP such that there was no detectable difference between CP-STINGΔTM and CP-STINGΔTMΔC9, the latter of which cannot activate the STING signaling. In addition to tumor size measurement for therapeutic efficacy, we further measured proinflammatory cytokines in a separate cohort of mice bearing the same tumor cells. The treatment group of “CP-STINGΔTM+cGAMP” displayed increased expression of CXCL10, TNFα and IFNγ, in comparison to “STINGΔTM+cGAMP” and the untreated group (FIGS. 6F and 6G).

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A composition, comprising a fusion protein and a STING agonist, wherein the fusion protein comprises STINGΔTM protein fused to a cell-penetrating domain or a nanobody.

2. The composition of claim 1, wherein the STINGΔTM comprises an amino acid sequence with at least 75%, 80%, 85%, 90%, 95%, 96%, 97%, 98%, 99% or 100% homology to the amino acid sequence selected from SEQ ID NOs: 3-6.

3. The composition of claim 1, wherein the cell-penetrating domain or the nanobody is fused to the N-terminus of the STINGΔTM.

4. The composition of claim 1, wherein the cell-penetrating domain comprises an amino acid sequence selected from SEQ ID NOs: 7-42.

5. The composition of claim 1, wherein the nanobody is capable of binding to a cancer cell.

6. The composition of claim 5, wherein the nanobody is capable of binding to CTLA4, PD-1, PD-L1, PD-L2, A2AR, B7-H3, B7-H4, BTLA, KIR, LAG3, TIM-3 or VISTA.

7. The composition of claim 1, wherein the STING agonist is a cytosolic cyclic dinucleotide (CDN).

8. The composition of claim 7, wherein the CDN is c-di-GMP, 3′,3′cGAMP, 2′,3′cGAMP, c-di-AMP, cAIMP, cAIMP Difluor, cAIM(PS)2 Difluor (Rp,Sp), 2′2′-cGAMP, 2′3′-cGAM(PS)2 (Rp,Sp), 3′3′-cGAMP Fluorinated, c-di-AMP Fluorinated, 2′3′-c-di-AMP, 2′3′-c-di-AM(PS)2 (Rp,RP), 2′3′-c-di-AM(PS)2, c-di-GMP Fluorinated, 2′3′-c-di-GMP, or c-di-IMP.

9. The composition of claim 1, wherein the STING agonist is a non-nucleotidyl small molecule.

10. The composition of claim 9, wherein the non-nucleotidyl small molecule is 5,6-dimethylxanthenone-4-acetic acid 7 (DMXAA), flavone-8-acetic acid, 2,7-bis(2-diethylamino ethoxy)fluoren-9-one, 10-carboxymethyl-9-acridanone, 2,7,2″,2″-dispiro[indene-1″,3″-dione]-tetrahydro dithiazolo[3,2-a:3′,2′-d]pyrazine-5,10(5aH,10aH)-dione, 4-(2-chloro-6-fluorobenzyl)-N-(furan-2-yl methyl)-3-oxo-3,4-dihydro-2H-benzo[b][1,4]thiazine-6-carboxamide, 6-Bromo-N-(naphthalen-1-yl)benzo[d][1,3]dioxole-5-carboxamide, 3-oxo-3,4-dihydro-2H-benzo[b][1,4]thiazine-6-carboxamide, 2-oxo-2,3-dihydro-1H-pyrido[2,3-b][1,4]thiazine-7-carboxamide, 2-oxo-1,2,3,4-tetrahydroquinoline-7-carboxamide, or 2-Oxo-1,2,3,4-tetrahydroquinazoline-7-carboxamides.

11. A fusion protein, comprising STINGΔTM protein fused to a cell-penetrating domain or a nanobody.

12. The fusion protein of claim 11, wherein the STINGΔTM comprises an amino acid sequence with at least 75%, 80%, 85%, 90%, 95%, 96%, 97%, 98%, 99% or 100% homology to the amino acid sequence selected from SEQ ID NOs: 3-6.

13. The fusion protein of claim 11, wherein the cell-penetrating domain or the nanobody is fused to the N-terminus of the STINGΔTM.

14. The fusion protein of claim 11, wherein the cell-penetrating domain comprises an amino acid sequence selected from SEQ ID NOs: 7-42.

15. The fusion protein of claim 11, wherein the nanobody is capable of binding to a cancer cell.

16. The fusion protein of claim 15, wherein the nanobody is capable of binding to CTLA4, PD-1, PD-L1, PD-L2, A2AR, B7-H3, B7-H4, BTLA, KIR, LAG3, TIM-3 or VISTA.

17. A nucleic acid molecule that hybridizes, under stringent conditions, with the complement of a nucleic acid encoding the fusion protein of claim 11.

18. A vector comprising the nucleic acid of claim 17.

19. A method of treating cancer or an infectious disease, comprising administering to a patient in need thereof an effective amount of a composition comprising a fusion protein and a STING agonist, wherein the fusion protein comprises STINGΔTM protein fused to a cell-penetrating domain or a nanobody.

20. The method of claim 19, wherein the STINGΔTM comprises an amino acid sequence with at least 75%, 80%, 85%, 90%, 95%, 96%, 97%, 98%, 99% or 100% homology to the amino acid sequence selected from SEQ ID NOs: 3-6.

21.-37. (canceled)
